(19) United States
(12) Patent Application Publication
(10) Pub. No.: US 2020/0399677 A1
(43) Pub. Date: Dec. 24, 2020

(54) METHODS FOR DIFFERENTIALLY QUANTIFYING NUCLEIC ACIDS

(71) Applicant: ChromaCode, Inc., Carlsbad, CA (US)

(72) Inventors: Christopher MacDonald, Carlsbad, CA (US); Lucien Jacky, Carlsbad, CA (US); Aditya Rajagopal, Carlsbad, CA (US)

(21) Appl. No.: 16/911,268

(22) Filed: Jun. 24, 2020

Related U.S. Application Data
(60) Provisional application No. 62/865,832, filed on Jun. 24, 2019.
(60) Provisional application No. 62/888,964, filed on Aug. 19, 2019.
(60) Provisional application No. 62/888,966, filed on Aug. 19, 2019.

(51) Int. Cl. C12Q 1/683; C12Q 1/6876; C12Q 1/682; G01N 21/64; G01N 21/76

(52) U.S. Cl. CPC C12Q 1/683; C12Q 1/6876; C12Q 1/682; G01N 2021/6439; G01N 21/76; C12Q 2600/154; C12Q 2600/156; G01N 21/6428

(57) ABSTRACT

Methods for differentially quantifying nucleic acids in a biological sample using differentially methylated loci include forming a digested biological sample in a solution of a methylation-sensitive restriction enzyme to cleave an unmethylated group of nucleic acids, forming a reaction mixture, amplifying the reaction mixture to generate a first signal from the a group of detection probes and a second signal from a second group of detection probes, determining a ratio comprising a first value derived from the first signal to a second value derived from the second signal, and identifying the proportion of nucleic acid in the biological sample originating from the first plurality of nucleic acids.

### CROSS-REFERENCE TO RELATED APPLICATIONS

This application claims priority to U.S. Provisional Patent Application No. 62/865,832, filed on Jun. 24, 2019; U.S. Provisional Patent Application No. 62/888,964, filed on Aug. 19, 2019; and U.S. Provisional Patent Application No. 62/888,966, filed on Aug. 19, 2019, each of which are incorporated by reference in its entirety for all purposes.

### BACKGROUND

Detection and analysis of specific nucleic acid target(s) within a gene may reveal important information about the subject donor of the gene, such as the likelihood a subject may have a particular genetic disorder, or susceptibility of having a particular genetic disease. Generally, polymerase chain reaction (PCR) may be used to amplify nucleic acids for analysis. Digital PCR (dPCR) is one type of PCR that is useful for the detection and quantification of nucleic acids. Where two or more targets are present in a dPCR-based amplification, quantitative discrimination between the two or more targets using comparative fragment size, methylation state, and/or SNP detection can provide critical insights pertaining to the relative abundance of the targets in a sample. Traditional PCR methods are limited in their ability to efficiently discriminate between and quantify these multiple targets.

### SUMMARY

The technology disclosed herein is directed to methods and systems for differentially quantifying nucleic acids in a biological sample. In embodiments, differentially quantifying nucleic acids may include using differentially methylated loci. For example, in embodiments, a method for differentially quantifying nucleic acids using differentially methylated loci may include providing a solution of a methylation-sensitive restriction enzyme, providing a biological sample with a first plurality of nucleic acids and a second plurality of nucleic acids, and forming a digested biological sample by combining the biological sample and the solution. The first and second pluralities of nucleic acids may each include a group of methylated nucleic acids and a group of unmethylated nucleic acids. Forming the digested biological sample may involve cleaving the unmethylated group of nucleic acids with a methylation-sensitive restriction enzyme. The method may also include forming a reaction mixture by combining the digested biological sample with, for example a set of amplification oligomers configured to amplify the first and second pluralities of nucleic acids, a first group of detection probes configured to anneal to a region of the first plurality of nucleic acids, and a second group of detection probes configured to anneal to a region of the second plurality of nucleic acids.

In some examples, the method includes amplifying the reaction mixture to generate a first signal from the first group of detection probes and a second signal from the second group of detection probes; determining a ratio including a first value derived from the first signal to a second value derived from the second signal, and identifying a proportion of nucleic acid in the biological sample originating from the first plurality of nucleic acids. In some embodiments, identifying the proportion of nucleic acid in the biological sample originating from the first plurality of nucleic acids further includes determining a fraction of methylated nucleic acids in the biological sample. In some embodiments, determining a fraction of methylated nucleic acids in the biological sample includes comparing the ratio to a reference value. In some embodiments, the methylation-sensitive restriction enzyme may include HpaII. In some embodiments, the methylation-sensitive restriction enzyme may include one or more of HpaII, AatII, AccII, Aor13HI, Aor51HI, BspT104I, BssHII, Cfr10I, ClaI, CpoI, Eco52I, HaeII, HhaI, MluI, NaeI, NotI, NruI, NsbI, PmaCI, Psp1406I, PvuI, SacII, SalI, SmaI and SnaBI.

In some embodiments, the method may include partitioning the reaction mixture into reaction volumes. In some embodiments, partitioning the reaction mixture into reaction volumes is performed prior to amplification. In some embodiments, the first and second signals each may include cumulative signal measurements generated from a total number of amplified reaction volumes containing an amplicon from each of the first and second pluralities of nucleic acids. In some embodiments, the first and second signals each may include cumulative signal measurements generated from each of the first and second pluralities of nucleic acids, wherein the reaction volumes are not quantified. In some embodiments, the first signal and the second signal are cumulative signal measurements generated from a total number of amplified reaction volumes containing one positive amplicon from each of the first and the second pluralities of nucleic acids. In embodiments, the first signal and the second signal may be generated in a single fluorescence channel. In some embodiments, the first signal and the second signal may be generated in more than one fluorescence channels. In some embodiments, the reaction mixture may also include buffers, reagents, a thermostable polymerase, and/or deoxyribonucleotide triphosphates.

In some embodiments, the set of amplification oligomers may include a forward amplification oligomer and a reverse amplification oligomer. In some embodiments, the set of amplification oligomers may bind to and amplify a region of the first plurality of nucleic acids and a region of the second plurality of nucleic acids. In some embodiments, more than one set of amplification oligomers may be used concurrently. For example, a first set of amplification oligomers may be used to amplify a first plurality of nucleic acids. Similarly, in some embodiments, a second set of amplification oligomers may be used to amplify a second plurality of nucleic acids.

In embodiments, the biological sample may include genomic DNA from one or more organism. For example, the biological sample may include whole blood from a pregnant woman. In some embodiments, the whole blood may include both maternal nucleic acids and fetal nucleic acids. The first plurality of nucleic acids may include fetal nucleic acid sequences and the second plurality of nucleic acids may include maternal nucleic acid sequences.

In embodiments, the first group of detection probes and the second group of detection probes each further may include a detectable label selected from the group consisting of a chemiluminescent label, a fluorescent label, or any combination thereof. In some embodiments, the first group of detection probes and the second group of detection probes may each include a quencher. The ratio may include a number of nucleic acids containing both the first and second pluralities of nucleic acids. The ratio may be determined without quantifying the first and second pluralities of nucleic acids. In some embodiments, the ratio may be compared to a predetermined reference value in order to generate a quantity.

In embodiments, the first group of detection probes may be configured to detect a region of nucleic acid associated with fetal aneuploidy. For example, the region may include a region of chromosome 22, chromosome 21, chromosome 18, chromosome 13, chromosome 9, chromosome 8, and/or X chromosome.

Some embodiments of the present disclosure provide a kit for differentially quantifying nucleic acids in a biological sample using differentially methylated loci. The kit may include a methylation-sensitive restriction enzyme, a set of amplification oligomers configured to amplify a first plurality of nucleic acids and a second plurality of nucleic acids, a first group of detection probes configured to anneal to the first plurality of nucleic acids, and a second group of detection probes configured to anneal to the second plurality of nucleic acids. In some examples, the set of amplification oligomers may include a forward amplification oligomer and a reverse amplification oligomer. The first group of detection probes and the second group of detection probes may each contain a detectable label. In some embodiments, the kit may include buffers, reagents, a thermostable polymerase, and/or deoxyribonucleotide triphosphates. The detectable label may be selected from the group consisting of a chemiluminescent label, a fluorescent label, or any combination thereof. In some examples, the first group of detection probes and the second group of detection probes may each include a quencher.

In embodiments, a kit for differentially quantifying a fetal fraction in a biological sample using differentially methylated loci is provided. The kit may include a methylation-sensitive restriction enzyme, a set of amplification oligomers configured to amplify a plurality of fetal nucleic acids and a plurality of maternal nucleic acids, a first group of detection probes configured to anneal to the first plurality of fetal nucleic acids, and a second group of detection probes configured to anneal to the second plurality of maternal nucleic acids. In some examples, the set of amplification oligomers may include a forward amplification oligomer and a reverse amplification oligomer, wherein the set of amplification oligomers may amplify a region of the first and second pluralities of nucleic acids. The kit may also contain more than one group of amplification oligomers that may amplify other regions of the first and second pluralities of nucleic acids. The first group of detection probes and the second group of detection probes may each include a detectable label. In some embodiments, the detectable label may be a chemiluminescent label, a fluorescent label, or any combination thereof. In some embodiments, the first group of detection probes and the second group of detection probes may each include a quencher. In some examples, the kit may include buffers, reagents, a thermostable polymerase, and/or deoxyribonucleotide triphosphates. In some embodiments, the methylation-sensitive restriction enzyme may include HpaII, AatII, AccII, Aor13HI, Aor51HI, BspT104I, BssHII, Cfr10I, ClaI, CpoI, Eco52I, HaeII, HhaI, MluI, NaeI, NotI, NruI, NsbI, PmaCI, Psp1406I, PvuI, SacII, SalI, SmaI or SnaBI.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 illustrates by way of example, differential methylation-sensitive restriction enzyme digestion and subsequent amplification in accordance with various embodiments disclosed herein.

FIG. 2 is an operational flow diagram illustrating an example method for differentially quantifying nucleic acids using methylation-sensitive restriction enzymes, in accordance with implementations of the disclosure.

FIG. 3 is an operational flow diagram illustrating an example method for differentially quantifying nucleic acids using fragment size distribution, in accordance with implementations of the disclosure.

FIG. 4A illustrates by way of example, differential quantification via fragment size distribution in accordance with various embodiments disclosed herein.

FIG. 4B illustrates by way of example, differential quantification via fragment size distribution in accordance with various embodiments disclosed herein.

FIG. 5 is an operational flow diagram illustrating an example method for differentially quantifying nucleic acids single nucleotide polymorphisms, in accordance with implementations of the disclosure.

FIG. 6 illustrates a computer component that can be utilized in implementing architectures and methods, in accordance with various implementations of the disclosure.

FIG. 7 illustrates, by way of example, a simulation using fragment size distribution in accordance with various embodiments of the disclosure.

FIG. 8 illustrates, by way of example, a simulation using fragment size distribution in accordance with various embodiments of the disclosure.

FIG. 9 illustrates, by way of example, using fragment size distribution for non-invasive prenatal testing applications in accordance with various embodiments of the disclosure.

FIG. 10 illustrates, by way of example, a simulation using single nucleotide polymorphisms in accordance with various embodiments of the disclosure.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION

The following description provides specific details for a comprehensive understanding of, and enabling description for, various embodiments of the technology. It is intended that the terminology used be interpreted in its broadest reasonable manner, even where it is being used in conjunction with a detailed description of certain embodiments.

The technology disclosed herein is directed to methods and systems for differentially quantifying nucleic acids in a biological sample using Polymerase Chain Reaction (PCR) technology and by using the inherent differences that exist between the nucleic acids including their comparative fragment size, methylation status, and/or single nucleotide polymorphisms (SNPs). PCR is a method of exponential amplification of specific nucleic acid target in a reaction mix with a nucleic acid polymerase and primers. Primers are short single stranded oligonucleotides which are complementary to the 3′ sequences of the positive and negative strand of the target sequence. The reaction mix is cycled in repeated heating and cooling steps. The heating cycle denatures or splits a double stranded nucleic acid target into single stranded templates. In the cooling cycle, the primers bind to complementary sequence on the template. After the template is primed the nucleic acid polymerase creates a copy of the original template. Repeated cycling exponentially amplifies the target 2-fold with each cycle leading to approximately a billion-fold increase of the target sequence in 30 cycles.

Digital PCR (dPCR) is a process of partitioning a sample containing one or more targets into a plurality of partitions (e.g., wells, droplets, etc.), performing a PCR reaction in each partition, and recording the luminescence (e.g., fluorescence) generated by, for example, a target-specific reporter probe. The use of labeled oligonucleotide probes enables specific detection. Digital PCR is generally performed on a digital PCR instrument that measures the fluorescence from each partition in an optical channel through one or more excitation/emission filter sets. Digital PCR is not limited to fluorescence and may be used in a variety of nucleic acid detection methods.

Primers, or “amplification oligomers,” used herein interchangeably, refer to an oligonucleotide or nucleic acid configured to bind to another nucleic acid and facilitate one or more reactions, for example, transcription, nucleic acid synthesis, and nucleic acid amplification. A primer may be single-stranded. A primer may be a forward primer or a reverse primer. A forward primer and a reverse primer may be those which bind to opposite strands of a double-stranded nucleic acid. For example, a forward primer may bind to a region of a first strand (e.g., Watson strand) derived from a nucleic acid, and a reverse primer may bind to a region of a second strand (e.g., Crick strand) derived from the nucleic acid. A forward primer may bind to a region closer to the start site of a gene relative to a reverse primer or may bind closer to the end site of a gene relative to a reverse primer. A forward primer may bind to the coding strand of a nucleic acid or may bind to the non-coding strand of a nucleic acid. A reverse primer may bind to the coding strand of a nucleic acid or may bind to the non-coding strand of a nucleic acid.

Target-specific oligonucleotide probe(s), also known as “detection probes” herein, is/are a short oligonucleotide(s) exhibiting complementary to one strand of the amplified target. The probe lacks a 3′ hydroxyl and therefore is not extendable by the DNA polymerase. TaqMan® (ThermoFisher Scientific) chemistry is a common reporter probe method used for multiplex Real-Time PCR. The TaqMan oligonucleotide probe may be covalently modified with a fluorophore and a quenching tag (i.e., a quencher). In this configuration the fluorescence generated by the fluorophore may be quenched and may not detected by the real time PCR instrument. When the target of interest is present, the probe oligonucleotide may base pair with the amplified target. While bound, it may be digested by the 5′ to 3′ exonuclease activity of the Taq polymerase thereby physically separating the fluorophore from the quencher and liberating signal for detection by the real time PCR instrument.

Multiplex analysis of multiple nucleic acid targets in a single measurement may be performed by encoding each nucleic acid target to a unique intensity value or range of values. For example, when detecting multiple nucleic acid targets in a sample using a single measurement, oligonucleotide probes may be provided at varying concentrations, such that the intensity of each signal generated from the probes, both individually and in combination, is unique. The signal may also be similar to those of other amplicons in the reaction where multiple targets are set to the same intensity level.

The term “channel,” “color channel,” or “optical channel”, as used herein, refers to a range of wavelengths. The range of wavelengths may be set or determined based on particular filters, which remove or “filter out” particular wavelengths. The terms “channel,” “color channel,” and “optical channel” may be used interchangeably.

Provided herein are methods for differentially quantifying nucleic acids in a biological sample. In embodiments, the present disclosure provides methods for unambiguously detecting the presence or absence of a nucleic acid target in a sample that may include a plurality of nucleic acids. For example, in embodiments, differentially quantifying nucleic acids may include using differentially methylated loci. In some embodiments, methods for the enrichment and quantification of a nucleic acid target in a biological sample may include using a dPCR reaction coupled with methylation-sensitive restriction enzymes (MSREs) to differentially cleave hyper-methylated sites on nucleic acids prior to amplification. For example, a certain plurality of nucleic acids that may include unmethylated cell-free DNA (i.e., cfDNA), may be mixed with an unknown quantity of hyper-methylated DNA. In embodiments, other PCR applications, including for example qPCR, may also be used.

In some embodiments, differentially quantifying nucleic acids in a biological sample may include using comparative fragment size distribution. For example, the present disclosure provides methods for increasing the accuracy of comparative quantification of multiple nucleic acid targets in a sample including a plurality of nucleic acids in a PCR reaction by constructing an assay consisting a plurality of probes, which in some embodiments may be contiguous, wherein a central probe of a short fragment size may be configured to anneal to a first nucleic acid target, and wherein the central probe is flanked between two additional probes configured to anneal to a second nucleic acid target, wherein the second nucleic acid target may, or may not, include the specific nucleotide sequence of the first nucleic acid target, thereby providing a fluorescent ratio of the relative abundance of the first and the second target nucleic acids.

Some embodiments of the present disclosure provide methods for increasing the accuracy of comparative quantification of multiple nucleic acid targets in a sample in a PCR reaction by selective enrichment using capture oligos exhibiting complementarity to polynucleotide regions flanking the nucleic acid target region of a nucleic acid target in the sample of mixed DNA.

In some embodiments, differentially quantifying nucleic acids in a biological sample may include using single nucleotide polymorphisms (SNPs). For example, a method for estimating fragment size of a nucleic acid target in a sample of nucleic acids using dPCR (and/or qPCR) may include encoding multiple SNPs in each detection channel and quantifying the relative ratios based on idealized SNP distributions. The various methods described herein may be also be implemented through use of a kit.

The various methods and systems described herein may be used in several applications, including, for example, in non-invasive prenatal testing (NIPT), where the risk that a fetus will be born with certain genetic abnormalities is examined. A pregnant female's blood contains both maternal DNA and certain levels of cell-free fetal DNA, or DNA from the placenta that has crossed into the mother's bloodstream. The methods and systems described herein may be used to detect and measure the circulating fetal DNA in order to determine whether the fetus will be born with a genetic abnormality. For example, the methods and systems disclosed herein may be used to determine whether the fetus displays high amounts of chromosome 21, 13, or 18, which may suggest that the fetus may have an extra copy of one of those chromosomes in each cell in the body, rather than the usual two copies. Having an extra 21 chromosome, for instance, can lead to Down Syndrome, a disorder characterized by intellectual and physical disabilities, including cognitive delays, heart defects, and an increased risk of developing Alzheimer's disease.

Differential Quantification Using Differentially Methylated Loci

Methylation-sensitive restriction enzymes (MSREs) may be used to differentiate between one or more nucleic acid targets within a biological sample. MSREs are restriction endonucleases that are sensitive to the methylation status of the bases in the recognition motif and differentially cleave a site based on that methylation status. Examples of MSREs that may be used in the methods and systems described herein include, but are not limited to, HpaII, AatII, AccII, Aor13HI, Aor51HI, BspT104I, BssHII, Cfr10I, ClaI, CpoI, Eco52I, HaeII, HhaI, MluI, NaeI, NotI, NruI, NsbI, PmaCI, Psp1406I, PvuI, SacII, SalI, SmaI and SnaBI, among others.

DNA methylation is an important epigenetic process. During DNA replication, a methyl group is added on the pyrimidine ring of cytosine on the 5 position (5-methylcytosine) in methylation (CpG cytosines). MSREs are restriction endonucleases which are sensitive to the methylation status of the bases in the recognition motif and differentially cleave a site based upon that methylation status of cytosine residues in CpG sequences. Because these restriction enzymes are unable to cleave methylated-cytosine residues, methylated DNA is left intact following digestion, while the unmethylated-cytosine DNA fragments are digested. Methylated DNA may therefore be enriched and selected for following MSRE digestion, and subsequent amplification and detection.

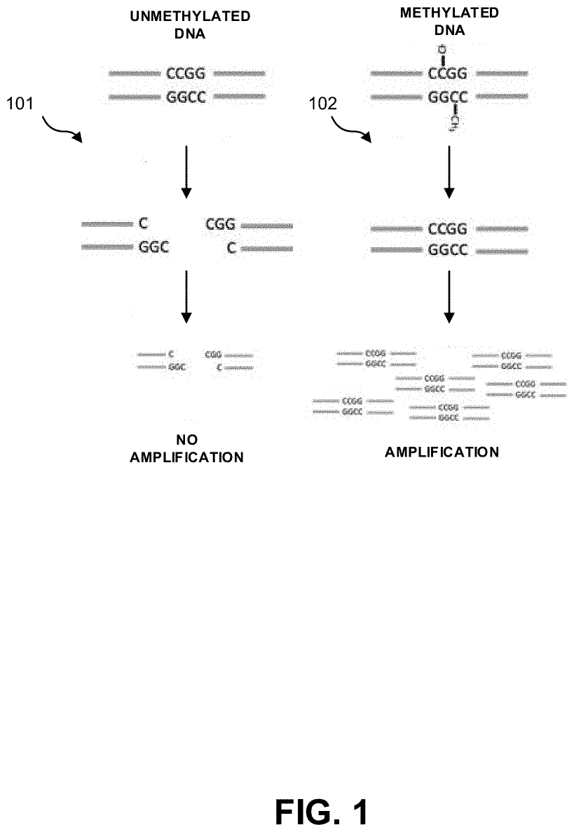

FIG. 1 illustrates by way of example, differential MSRE digestion and subsequent amplification in accordance with various embodiments disclosed herein. Cleaved DNA 101 includes unmethylated DNA that has been digested via MSREs prior to amplification. Un-cleaved DNA 102 remains intact following digestion with MSREs due to the methylation status of the cytosine residues. Following digestion, un-cleaved DNA 102 may be amplified and enriched for using, for example, dPCR applications. In some embodiments, other PCR applications (e.g., qPCR) may also be used

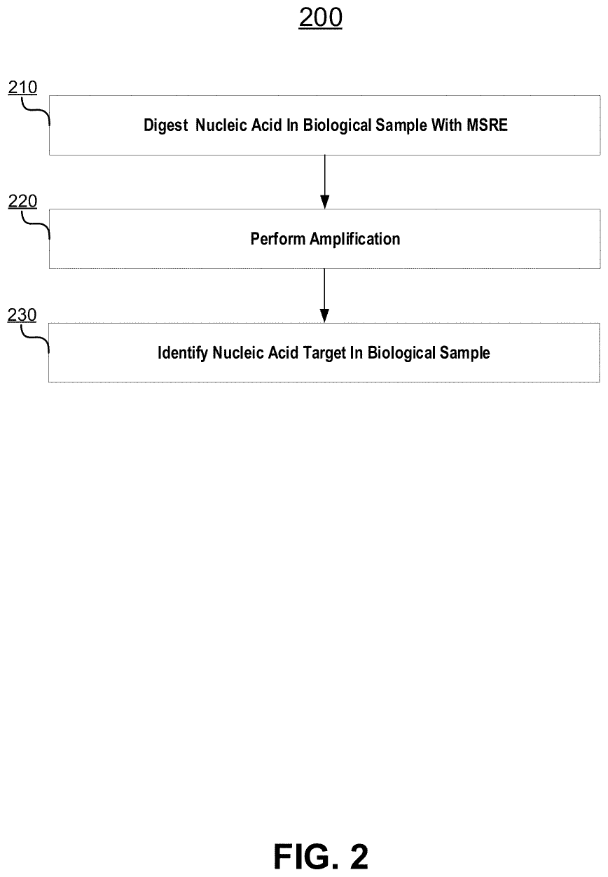

FIG. 2 is a flow diagram illustrating an example method for differentially quantifying nucleic acids using methylation-sensitive restriction enzymes. At a high level, method 200 may be performed to differentially quantify nucleic acids in accordance with various embodiments of the disclosure. In embodiments, method 200 provide a means for differentially quantifying a first plurality of nucleic acids from a second plurality of nucleic acids using MSRE. The operations of the various methods described herein are not necessarily limited to the order described or shown in the figures, and one of skill in the art will appreciate, upon studying the present disclosure, variations of the order of the operations described herein that are within the spirit and scope of the disclosure. Let it be appreciated that operations of method 200 may be performed multiple times.

At operation 210, the first and second pluralities of nucleic acids in a biological sample are digested by the MSRE. In embodiments, the first and second pluralities of nucleic acids may be derived from more than one organism, wherein each organism contributes a plurality of nucleic acids. In some embodiments, the first and second pluralities of nucleic acids may be derived from more than one source. In some embodiments, the first and second pluralities of nucleic acids may be derived from a single source. For example, in some embodiments, the biological sample may include the whole blood of a pregnant female. In some embodiments, a first plurality of nucleic acids may include fetal nucleic acids, including for example, cell free fetal DNA. In some embodiments, a second plurality of nucleic acids may include maternal nucleic acids, including for example, cell free maternal DNA. In some embodiments, method 200 may be used to differentially quantify a fetal nucleic acid target and a maternal nucleic acid target in a biological sample. In embodiments, the first plurality of nucleic acids may be derived from the fetal nucleic acid target, and a second plurality of nucleic acids may be derived from the maternal nucleic acid target.

At operation 210, the first and second pluralities of nucleic acids may be digested. In some embodiments, digestion during operation 210 may include a solution including a methylation-sensitive restriction enzyme (MSRE), for example, HpaII, wherein the solution is combined with the biological sample including a first and second plurality of nucleic acids. In some embodiments, the solution and the biological sample may be combined prior to PCR amplification in order to cleave the unmethylated DNA, whilst leaving the methylated DNA from intact.

For example, operation 210 may include pretreatment of cell free DNA with a methylation-sensitive restriction enzyme such as HpaII prior to PCR amplification to cleave the unmethylated DNA, whilst leaving the methylated cell free DNA intact. In embodiments, the MSRE may be deactivated and/or denatured following digestion. For example, the MSRE may be deactivated or denatured through the application of heat (i.e., heat killed). In some embodiments, deactivation of the MSRE occurs prior to PCR amplification. In some embodiments, deactivation of the MSRE occurs prior to forming the reaction mixture. It is to be understood that the biological sample may include DNA and/or cDNA. The biological sample may be derived from more than one organism. In some embodiments, the biological sample may be derived from blood, sweat, urine, tears, or any other bodily secretion. Moreover, in some embodiments, the biological sample may be obtained from a human or from some other animal.

Following digestion of the biological sample, in some embodiments, a reaction mixture may be formed with a set of amplification oligomers configured to amplify the first and second pluralities of nucleic acids (spanning the MSRE recognition motif of both first and second nucleic acid target); a first group of detection probes configured to anneal to a region of the first plurality of nucleic acids; and a second group of detection probes configured to anneal to a region of the second plurality of nucleic acids. In some embodiments, the first plurality of nucleic acids may include fetal nucleic acid sequences, wherein the plurality of nucleic acids include genetic material of the fetus. In some embodiments, the second plurality of nucleic acids may include maternal nucleic acid sequences, wherein the plurality of nucleic acids include genetic material of the pregnant female. In some embodiments, the nucleic acid sequence amplified may include a region of nucleic acids associated with fetal aneuploidy. For example, in embodiments, the region may include a region of chromosome 22, chromosome 21, chromosome 18, chromosome 13, chromosome 9, chromosome 8, or X chromosome. In some embodiments, the reaction mixture may further include one or more of buffers, reagents, a thermo stable polymerase, and deoxyribonucleotide triphosphates.

In embodiments, once the biological sample is digested and the reaction mixture formed, the reaction mixture may be partitioned into reaction volumes (e.g., wells, microwells, droplets, etc.). For example, in some embodiments, the amplification may be performed in a droplet in an emulsion. The amplification may be performed in a microwell. In some embodiments the sample may be partitioned into a plurality of reaction volumes.

At operation 220, the reaction mixture including the digested nucleic acid, is amplified. In some embodiments, the reaction mixture may be partitioned into reaction volumes and amplified. In some embodiments, more than one reaction volumes may be amplified simultaneously. In embodiments, once the reaction mixture is partitioned into reaction volumes, the reaction volume may then be subjected to PCR amplification whereby the set of amplification oligomers (spanning the MSRE recognition motif) will amplify the methylated DNA strands but not the unmethylated DNA strands. In embodiments, this method may be used in digital PCR and/or qPCR assays to enrich the fraction which is highly methylated DNA compared to hypomethylated DNA.

In some embodiments, operation 220 may include a set of amplification oligomers (i.e., a forward primer and a reverse primer) configured to amplify both the first and second pluralities of nucleic acids that are of a given length (e.g., may hybridize to regions of a nucleic acid sequences that are at a certain distance apart). For example, a set of amplification oligomers may be configured to amplify a first plurality of nucleic acids of a length of between 50 and 300 base pairs, or more. In some examples, a set of amplification oligomers may be configured to amplify a first plurality of nucleic acids of a length of about 50, 75, 100, 125, 150, 175, 200, 225, 250, 275, or 300 base pairs.

In some embodiments, operation 220 may include a set of amplification oligomers may be configured to amplify a second plurality of nucleic acids that may or may not be longer in length than the first plurality of nucleic acids. In some embodiments, a set of amplification oligomers may be configured to amplify a second plurality of nucleic acids that are longer in length (i.e., encompasses a greater number of nucleobases) than the first plurality of nucleic acids (e.g., may hybridize to regions of a nucleic acid sequence a given distance apart). In some embodiments, a set of amplification oligomers may be configured to amplify a second plurality of nucleic acids with a length of between 300 and 750 base pairs, or more. In some embodiments, the set of amplification oligomers may be configured to amplify second plurality of nucleic acids of a length of about 300, 325, 350, 375, 400, 425, 450, 500, 550, 600, 650, 700, 750 base pairs.

In embodiments, there may be more than one set of amplification oligomers that may differentially detect and amplify more than one plurality of nucleic acids.

In embodiments, operation 220 may include a first group of detection probes that may be configured to anneal to a region of the first plurality of nucleic acids of a given length. In some embodiments, the first group of detection probes may include a non-target hybridizing sequence. In some embodiments, the first group of detection probes may include a molecular beacon, a molecular torch, and/or a detectable label. In some embodiments, a detectable label may include a chemiluminescent label. In some embodiments, a detectable label may be include a fluorescent label. In some embodiments the first group of detection probes may include a quencher.

In embodiments, operation 220 may include a second group of detection probes may be configured to anneal to the second plurality of nucleic acids (that may or may not be longer in length than the first nucleic acid sequence). In some embodiments, the second group of detection probes may include a non-target hybridizing sequence. In some embodiments, the second group of detection probes may include a molecular beacon, a molecular torch, and/or a detectable label. In some embodiments, a detectable label may include a chemiluminescent label. In some embodiments, a detectable label may include a fluorescent label. In some embodiments the second group of detection probes may include a quencher.

In embodiments, the first group of detection probes may be configured to anneal to the first plurality of nucleic acids, which in some embodiments may include fetal nucleic acid sequences. In some embodiments, the first group of detection probes may be configured to anneal to and detect a region of fetal nucleic acid sequences. In some embodiments, the first group of detection probes may be configured to anneal to and detect a region of fetal nucleic acid sequences, wherein the region is associated with fetal aneuploidy.

In embodiments, the second group of detection probes may be configured to anneal to the second plurality of nucleic acids, which in some embodiments, may include maternal nucleic acid sequences. In some embodiments, the second group of detection probes may be configured to anneal to and detect a region of maternal nucleic acid sequences. In some embodiments, the second group of detection probes may be configured to anneal to and detect a region of maternal nucleic acid sequences, wherein the region is associated with fetal aneuploidy.

Following amplification, target nucleic acid may be identified at operation 230. In embodiments, operation 230 may involve use of a signal. In embodiments, amplification of the reaction volume results in the generation of a first signal from the first group of detection probes and a second signal from a second group of detection probes. In some embodiments, the first and second signal each may each include cumulative signal measurements generated from the total number of amplified reaction volumes containing an amplicon from each of the first and second pluralities of nucleic acids. In some embodiments, the first and second signals each may include cumulative signal measurements generated from each of the first and second pluralities of nucleic acids, wherein the reaction volumes are not quantified. In some embodiments, the signals from the respective detection probes may be used to determine a ratio. In embodiments, a ratio may include a first value derived from the first signal to a second value derived from the second signal. In some embodiments, the ratio may include a first value from the first signal to a second value from the second signal, wherein the values from the respective signals do not include a quantified sum generated from the total number of amplified reaction volumes.

In some embodiments, the first signal may be generated in a single fluorescent channel. In some embodiments, the first signal may be generated in more than one fluorescent channel. In some embodiments, the second signal may be generated in a single fluorescent channel. In some embodiments, the second signal may be generated in more than one fluorescent channel. In some embodiments, the first and second signal may be generated in a single fluorescent channel. In some embodiments, the first and second signal may be generated in more than one fluorescent channels.

In embodiments, operation 230 may involve identifying the proportion of nucleic acids in the biological sample originating from the first plurality of nucleic acids. In some embodiments, identifying the proportion of nucleic acids in the biological sample originating from the first plurality of nucleic acids may include determining a fraction of methylated nucleic acids in the biological sample. In some embodiments, determining a fraction of methylated nucleic acids in the biological sample may include comparing the ratio to a reference value. In embodiments, the reference value may be previously determined before performing the instant method. For example, the reference value may represent a known value or quantity (e.g., a standard) by which the ratio may be compared to. The reference value may be obtained from a different DNA quantifying method or machine, for example a spectrophotometer. Comparing the ratio to a reference value may itself be performed via a machine (e.g., computer implemented). In embodiments, operation 230 may involve comparing the proportion of nucleic acids that are methylated to the proportion of nucleic acids that are unmethylated. In some embodiments, operation 230 may include identifying specific regions of nucleic acid sequences that are differentially methylated to other specific regions that are homogenously methylated.

The methods and systems disclosed herein may be employed in various systems including, for example, non-invasive prenatal testing (NIPT). NIPT is an important method of determining the risk that a fetus will be born with certain genetic abnormalities. However, because NIPT measurements count the fragments of circulating fetal DNA, testing is often hindered by the inability to capture enough circulating fetal DNA to examine. However, by identifying sites that are hyper-methylated in the placenta DNA, methods and systems disclosed herein may be used to differentially detect fetal cfDNA. Accordingly, the technology disclosed herein may be used to enrich the fetal fraction in order to reduce the total number of chromosomal counts required for a high confidence call of trisomy in the maternal background.

The various methods and systems disclosed herein may also be implemented through use of a kit. For example, in embodiments, a kit for differentially quantifying nucleic acids in a biological sample using differentially methylated loci may include a methylation-sensitive restriction enzyme, for example HpaII. In some embodiments, the kit may also include a set of amplification oligomers configured to amplify a first plurality of nucleic acids and a second plurality of nucleic acids. The kit may also include a first group of detection probes configured to anneal to the first plurality of nucleic acids and a second group of detection probes configured to anneal to the second plurality of nucleic acids. In some embodiments, the set of amplification oligomers may include a forward amplification oligomer and a reverse amplification oligomer. In some embodiments, there may be more than one set of amplification oligomers that may be configured to detect differentially between more than one plurality (i.e., pluralities) of nucleic acids. In some embodiments, the first group of detection probes may include a detectable label. In some embodiments, the second group of detection probes may include a detectable label. In embodiments, the kit may be used to select for and enrich certain target nucleic acids using the various methods disclosed herein.

Differential Quantification Using Fragment Size Distribution

In embodiments, fragment size distribution may be used to differentiate between one or more nucleic acid targets within a biological sample. Indeed, targeted amplification (and subsequent differentiation) of nucleic acids based on nucleotide length is a useful tool in molecular diagnostics. The present disclosure also provides for methods and systems for detecting and quantifying target nucleic acids in a biological sample based on nucleotide length. The methods described herein may be used to determine whether the target nucleic acid sequences originates from a single source or from two or more sources. For example, in embodiments, the methods disclosed herein for differentially quantifying nucleic acid using fragment size distribution may be used for certain applications, including, non-invasive prenatal testing. Fetal DNA is significantly shorter length than the background maternal DNA. Thus, it is advantageous that methods and systems described herein allow for analysis of only the fetal DNA or at a minimum an enhanced fraction of the DNA. Moreover, measuring the relative abundance of fragments of different sizes enables the ability to estimate the percentage of fetal DNA in the sample.

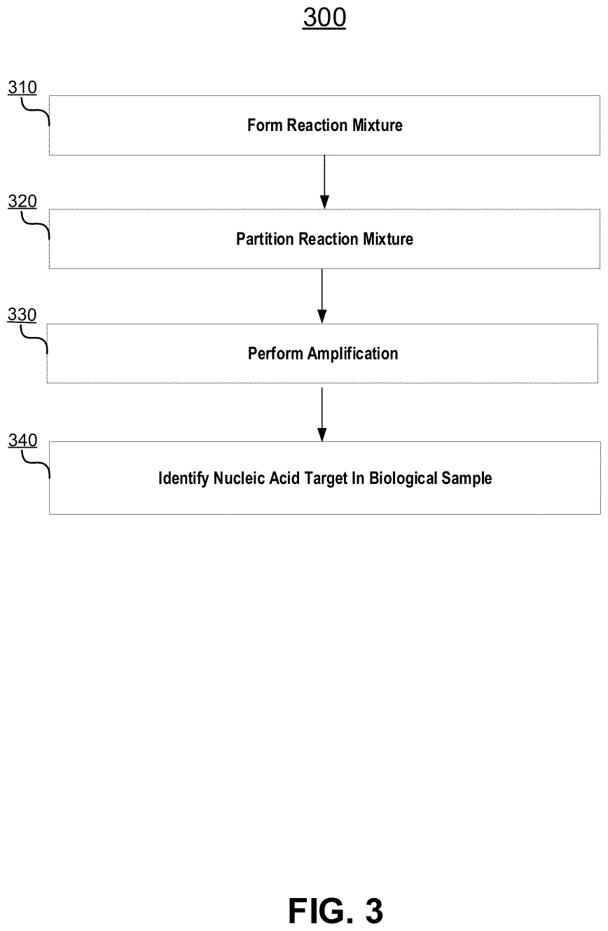

FIG. 3 is a flow diagram illustrating an example method for differentially quantifying nucleic acids using fragment size distribution. At a high level, method 300 may be performed to differentially quantify nucleic acids to increase the accuracy of the comparative quantification of two or more pluralities of nucleic acids (such as maternal cfDNA with an unknown fetal fraction) in a PCR reaction. In some embodiments, the method may include a plurality of detection probes, which in some embodiments may be contiguous, wherein a central detection probe of a short fragment size configured to anneal to a first nucleic acid target, and wherein the central detection probe is flanked between two additional detection probes configured to anneal to a second nucleic acid target. In embodiments, the second nucleic acid target may include the specific nucleotide sequence of the first nucleic acid target, thereby providing a fluorescent ratio of the relative abundance of the first and the second target nucleic acid. Let it be appreciated that operations of method 300 may be performed multiple times.

At operation 310, a reaction mixture is formed. The reaction mixture of operation 310 may include a biological sample including a first plurality of nucleic acids and a second plurality of nucleic acids. The first plurality of nucleic acids may include a first nucleic acid sequence and the second plurality of nucleic acids may include a second nucleic acid sequence that is longer than the first nucleic acid sequence. The first plurality of nucleic acids and the second plurality of nucleic acids may be a mix of contiguous or non-contiguous sequences that may be potentially from one or more separate organisms, but that also may be derived from a single source. Operation 310 may also include forming a reaction mixture by combining the biological sample with a first group of detection probes configured to anneal to a first region on the first and second nucleic acid sequences and a second group of detection probes configured to anneal to a second region on the second nucleic acid sequence. In some embodiments, the reaction mixture of operation 310 may include a first pair of amplification oligomers configured to amplify the first region on the first and second nucleic acid sequences, and a second pair of amplification oligomers configured to amplify the second region on the second nucleic acid sequence.

In embodiments, operation 310 may include forming a reaction mixture that includes a biological sample wherein the first plurality of nucleic acids may include a first fetal nucleic acid sequence. The second plurality of nucleic acids may include a second maternal nucleic acid sequence that is longer than the first fetal nucleic acid sequence. The first plurality of fetal nucleic acids and the second plurality of maternal nucleic acids may be a mix of contiguous or non-contiguous sequences. In some embodiments, operation 310 of method 300 may include forming a reaction mixture by combining the biological sample with a first group of detection probes configured to anneal to a first region on the fetal and maternal nucleic acid sequences, a second group of detection probes configured to anneal to a second region on the maternal nucleic acid sequence, a first pair of amplification oligomers configured to amplify the first region on the fetal and maternal nucleic acid sequences, and a second pair of amplification oligomers configured to amplify the second region on the maternal nucleic acid sequence

In embodiments, the reaction mixture of operation 310 may include buffers, reagents, a thermo stable polymerase, and/or deoxyribonucleotide triphosphates. In some embodiments, the first pair of amplification oligomers may include a first forward amplification oligomer and a first reverse amplification oligomer. In some embodiments, a second pair of amplification oligomers may include a second forward amplification oligomer and a second reverse amplification oligomer.

In some embodiments, the biological sample may include whole blood from a pregnant female. In some embodiments, the whole blood may include maternal nucleic acids and fetal nucleic acids. In some embodiments, the maternal nucleic acid may share a sequence with the fetal nucleic acid. In some embodiments, the first plurality of nucleic acid may include a region of nucleic acid associated with fetal aneuploidy. In some embodiments, the region may include a region of chromosome 22, chromosome 21, chromosome 18, chromosome 13, chromosome 9, chromosome 8, or X chromosome.

The first group of detection probes may be configured to anneal to the first region on the first and second nucleic acid sequences, and the second group of detection probes may be configured to anneal to the second region on the second nucleic acid sequence are contiguous. The first and second groups of detection probes may be contiguous or non-contiguous. In some embodiments, the first and second groups of detection probes may overlap.

At operation 320, method 300 may include partitioning the reaction mixture into a reaction volume. For example, operation 320 may include partitioning the reaction mixture into one or more reaction volumes. In embodiments, operation 320 may include partitioning the reaction mixture into a plurality of reaction volumes. Reaction volumes may include, for example, droplets in an emulsion, or reaction wells. In some embodiments, method 300 may be performed without partitioning the reaction mixture into reaction volumes.

At operation 330, method 300 may include performing an amplification reaction on the first nucleic acid sequence to generate a first signal from the first detection probe and the second nucleic acid sequence to generate a second signal from the second detection probe. The method may also include determining a ratio of a first value derived from the first signal to a second value derived from the second signal and identifying a size distribution by comparing the ratio to a reference value.

At operation 340, nucleic acid targets in the biological sample may be identified. For example, operation 340 may include determining a first signal from the first group of detection probes and a second signal from the second group of detection probes, thereby forming a ratio of a first value derived from the first signal to a second value derived from the second signal. The ratio may then be used to identify a fetal fraction by comparing it to a known reference value.

The present disclosure provides methods for comparative quantification of multiple nucleic acid targets in a biological sample. The biological sample may include a first plurality of nucleic acids and a second plurality of nucleic acids (for example, maternal cfDNA with an unknown fetal fraction). The methods may include a PCR reaction using multiple detection probes (e.g., more than one set of detection probes). The detection probes may be contiguous, wherein a first detection probe of a short fragment size may be configured to anneal to a first nucleic acid target. For example, the first detection probe may be flanked between two or more additional detection probes (i.e., second set of detection probes and/or a third set of detection probes, etc.) that are configured to anneal to a second nucleic acid target. The second nucleic acid target may include the specific nucleotide sequence of the first nucleic acid target, thereby providing a fluorescent ratio of the relative abundance of the first and the second target nucleic acid.

In some embodiments, the methods disclosed herein may be used in digital PCR (or other partition-based assay with multiple PCR sub-reactions) applications.

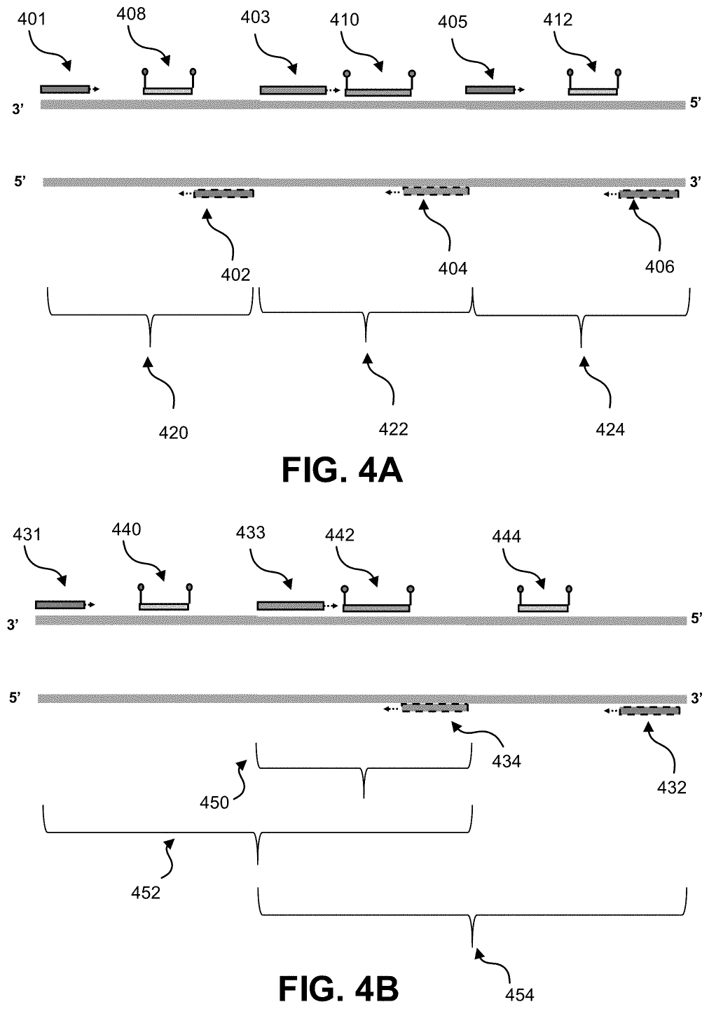

FIG. 4A and FIG. 4B illustrate by way of example, multiple sets of amplification oligomers/groups of detection probes for differential quantification via fragment size distribution in accordance with various embodiments disclosed herein. For example, in FIG. 4A, a first set of amplification oligomers 401 and 402 may bind and amplify a certain region, thereby producing fragment 420, which is of a certain size and/or length. In some embodiments, a second set of amplification oligomers 403 and 404 may bind and amplify a certain region, thereby producing fragment 422 that may or may not be of similar size to fragment 420. In some embodiments, a third set of amplification oligomers 405 and 406 may bind and amplify a certain region, thereby producing fragment 424 that may or may not be similar in size to fragments 420 and 422. In some embodiments, one or more groups of detection probes (e.g., detection probes 408, 410, and 412) may be used to produce a signal. FIG. 4B illustrates, by way of example, different configurations of amplification oligomers (e.g., 431 and 432; 433 and 434) and detection probes (e.g., 440 and 444) that may be used to obtain variations in the size of fragments 450, 452, and 454, in accordance with various implementations disclosed herein. Embodiments disclosed herein may also be without detection probes, but instead through use of, for example, intercalating dyes.

In some embodiments, the methods and systems disclosed herein may employ construction of a set of multiple amplification oligomers/detection probe sets as shown in FIGS. 4A-4B with a central detection probe of a short fragment size and flanking probe sets nearby. In some embodiments, the amplification oligomers/detection probe sets may be contiguous, and need not all have independent forward or reverse amplification oligomers, or detection probes.

Differential Quantification Using Single Nucleotide Polymorphisms

In embodiments, single nucleotide polymorphisms (SNPs) may be used to differentiate between one or more nucleic acid targets within a biological sample. SNPs involve the variation of a single nucleotide—adenine (A), guanine (G), thymine (T), or cytosine (C)—in a segment of a DNA molecule and may occur randomly throughout a genome. SNPs may occur as the result of a switch (i.e., substitution), removal (i.e., deletions) or addition (i.e., insertion) of a single nucleotide base within a polynucleotide sequence.

The methods and systems for differentially quantifying nucleic acids described herein may also be performed using SNPs. Similar to differential quantification based on methylation status and fragment size distribution, differential quantification using SNPs may also be employed in several applications, including in NIPT applications. For example, certain SNPs may exist that are exclusive to the fetal genome and, therefore, may be used to differentiate fetal nucleic acids from maternal nucleic acids. In embodiments, a fetal fraction may be determined from examining the ratio of fetal fragments to maternal fragments. In some embodiments, SNPs may be encoded into each channel, wherein the resulting ratio may be used to more accurately estimate the relative abundance of fetal DNA in a biological sample.

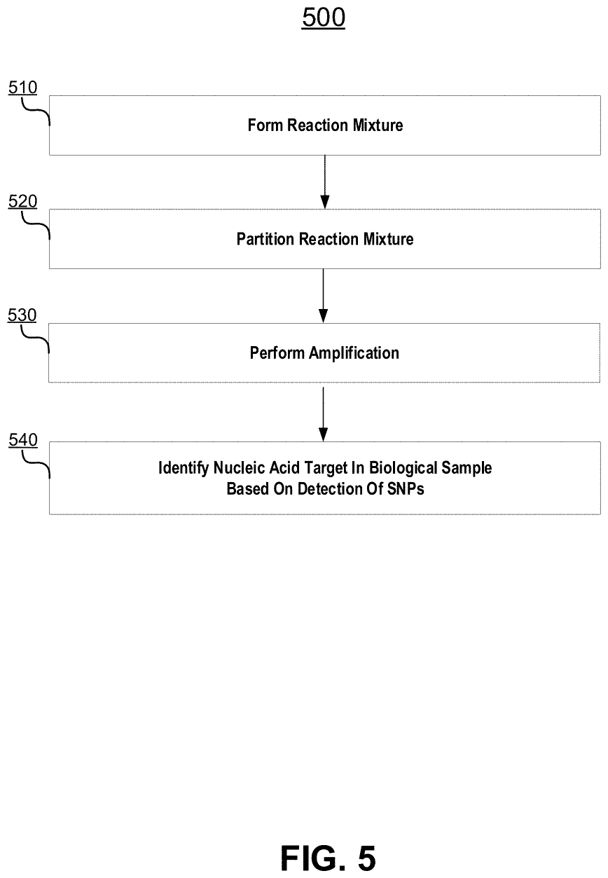

FIG. 5 is a flow diagram illustrating an example method for differentially quantifying nucleic acids using single nucleotide polymorphisms (SNPs). At a high level, method 500 may be performed to differentially quantify nucleic acids to increase the accuracy of the comparative quantification of two or more pluralities of nucleic acids (such as maternal cfDNA with an unknown fetal fraction) in a PCR reaction. Let it be appreciated that method 500 may be performed more than once.

At operation 510, a reaction mixture may be formed by combining a biological sample with a first set of amplification oligomers configured to amplify the first plurality of nucleic acids and a second set of amplification oligomers configured to amplify the second plurality of nucleic acids. The reaction mixture may also include a first group of detection probes configured to anneal to a single nucleotide polymorphism specific to the first plurality of nucleic acids and a second group of detection probes configured to anneal to a single nucleotide polymorphism specific to the second plurality of nucleic acids. In some embodiments, operation 510 may include generating a coding scheme for the first and second pluralities of nucleic acids.

In embodiments, the first and second groups of detection probes may each include different detectable labels. For example, the first group of detection probes may include a fluorophore of a first color, and the second group of detection probes may include a fluorophore of a second color. In some embodiments, the first and second groups of detection probes may each include an identical detectable label. Similarly, in some embodiments, the first and second groups of detection probes may each include a quencher. In some embodiments, the first and second groups of detection probes may beach include TaqMan® detection probes.

At operation 520, the reaction mixture may be partitioned into reaction volumes. In embodiments, the reaction mixture may be portioned into a reaction volume. In some embodiments, the reaction volume may include a droplet in an emulsion. In some embodiments, the reaction volume may be partitioned into a microwell, wherein amplification occurs in a microwell. In some embodiments, the reaction mixture may be partitioned into a plurality of reactions volumes. In some embodiments, method 500 may be performed without partitioning the reaction mixture into reactions volumes.

At operation 530, the reaction volume may be amplified. In some embodiments, amplifying the reaction volumes may generate a first sum signal from the first group of detection probes and a second sum signal from the second group of detection probes. In some cases, certain SNPs which are only in the fetal genome will amplify allowing quantitation of mean fetal counts in a well, and other SNPs which only are of maternal origin will amplify allowing quantitation of mean maternal counts in a well. In some embodiments, the ratio of these counts can be used in a calculation in order to estimate the fetal fraction.

At operation 540, a nucleic acid target may be identified using SNPs. In some embodiments, the method may include identifying the presence or absence of a genetic abnormality in the biological sample. In some embodiments, identifying the presence or absence of a genetic abnormality in the biological sample may include comparing the ratio to a reference value. In some embodiments, amplifying the reaction volume at operation 530 generates a first sum signal from the first group of detection probes and a second sum signal from the second group of detection probes.

In embodiments, operation 540 may include quantifying the first and second SNP targets may include determining a ratio of the first plurality of nucleic acid target to the second plurality of nucleic acid target in the sample (e.g., the quantity and abundance of the first nucleic acid target relative to the quantity and abundance of the second nucleic acid target in the biological sample). Quantifying SNPs targets may include determining an absolute quantity of the first and second targets in the sample. Quantifying SNP targets may include determining a relative quantity (i.e., relative ratio) of the first and second SNP targets in the sample. In some embodiments, the resulting ratio of these counts may be used in a calculation in order to estimate the fetal fraction. Determining the ratio of the first sum value to the second sum value may provide insight that can be used to calculate fetal fraction. In some embodiments, the ratio may be compared to a reference value. Comparing the ratio to the reference value may determine an estimated fetal fraction in a sample. The various methods for quantifying nucleic acids using SNPs disclosed herein may also be implemented through a kit.

It is to be understood that for the various methods and systems disclosed herein, nucleic acid target detection and differential quantification may be accomplished by the use of two or more reactions. For example, an assay for measuring a plurality of nucleic acid targets may include a first reaction and a second reaction. The results of the first and second reactions may together unambiguously detect the presence or absence of each of the nucleic acid targets. An assay may include any number of reactions, where the results of the reactions together identify a plurality of nucleic acid targets, in any combination of presence or absence. An assay may include two, three, four, five, six, seven, eight, nine, ten reactions, or more.

In some embodiments, the present disclosure provides a multiplexed assay for simultaneous amplification, detection, and or/quantification of an analyte in a sample. In some embodiments the methods of the disclosure may be used to detect and/or quantify 1, 2, 3, 4, 5, 6, 7, 8, 9, 10, 11, 12, 13, 14, 15, 16, 17, 18, 19, 20, or more analytes in a sample volume. In some embodiments, the present disclosure provides methods for performing a digital assay. A method for performing a digital assay may include partitioning a plurality of nucleic acid targets and a plurality of oligonucleotide probes into a plurality of reaction volumes. In some cases, two, three, four, five, or more nucleic acid targets may be partitioned into a plurality of partitions together with two, three, four, five, six, seven, eight, nine, ten, 11, 12, 13, 14, 15, 16, 17, 18, 19, 20 or more oligonucleotide probes. In some aspects, the disclosed method utilizes intercalating dyes, thereby removing the need for probes.

In some cases, a plurality of signals may be generated by one or more of the plurality of probes from the mixture. The plurality of signals may be generated by nucleic acid amplification of the plurality of nucleic acid molecules. Nucleic acid amplification may degrade the plurality of oligonucleotide probes (e.g., by activity of a nucleic acid enzyme), thereby generating the plurality of signals. A plurality of signals may be a plurality of fluorescent signals, a plurality of chemiluminescent signals, or a combination thereof.

In some aspects, the detection of the signals may be subjected to processing in order for the signals and data to be used for subsequent steps or downstream methods. The processing may use mathematical algorithms to analyze or process the signal data. In some cases, the mathematical algorithms used for data processing may include expectation maximization, nearest neighbor analysis, basic model parameterization, Bayesian estimation, or combinations thereof. The mathematical algorithm may use a process parameter. Examples or process parameters include parameters for threshold cycles, amplitudes, or slopes. In some case, the processing may use data obtained from the instrument or detector. Based on the detecting, the nucleic acid target in the sample may be quantified.

In some aspects, reference conditions may be used to quantify the relative ratios of multiple nucleic acid targets in a sample including a plurality of nucleic acids. As disclosed within, reference conditions may include, but are not limited to, a) a primer concentration, b) a polymerase concentration, c) polymerase type, d) a reference nucleic acid concentration, e) a number of thermocycles, f) a rate of thermocycling, g) a thermocycle time length, h) a probe sequence; i) a primer sequence, or combinations thereof. The reference condition with the known parameter may be used to extrapolate, interpolate or otherwise calculate a concentration, quantity, or amount of another nucleic acid in a separate sample. The generation of reference quantification conditions may be used to directly compare to generated quantification parameters of a data set or can be used to calculate a quantification parameter based on for example, parameterization, fitting, extrapolation, interpolation, or estimation of the data set or a parameter of the data set.

Methods as described herein may be performed without the use of immobilization, separation, mass spectrometry, or melting curve analysis. For example, identification of the analytes may be performed without obtaining a mass of the analytes via mass spectrometry or any similar technique. Additionally, the methods may be used without observing a melting reaction and plotting the signal against a temperature. For example, an analyte may be identified without subjecting the analyte to temperature gradient in order to analyze a specific temperature in which an analyte goes through a physical or chemical change. The methods as described herein may be corroborated via techniques using immobilization, separation, mass spectrometry, or melting curve analysis. For example, the melting curve may be used to verify a number of different amplicons or detecting a presence of an amplicon.

Digital Assays

In embodiments, the present disclosure provides methods for performing a digital assay. A method for performing a digital assay may include partitioning a plurality of nucleic acid targets and a plurality of oligonucleotide probes (i.e., detection probes) into a plurality of reaction volumes. In some cases, two, three, four, five, or more nucleic acid targets may be partitioned into a plurality of reaction volumes together with two, three, four, five, six, seven, eight, nine, ten, 11, 12, 13, 14, 15, 16, 17, 18, 19, 20 or more oligonucleotide probes. Following partitioning, the nucleic acid targets may be amplified in the reaction volumes, for example, by polymerase chain reaction (PCR). Next, N signals may be generated from the oligonucleotide probes. Each signal of the N signals may correspond to the presence of a unique combination of nucleic acid targets in a reaction volume. Following signal generation, the N signals may be detected in a single optical channel. The signals may be detected using, for example, fluorescence detection in a single-color channel.

Partitioning

Methods of the present disclosure may include partitioning a sample or sample into a plurality of reaction volumes. A sample may include nucleic acids, oligonucleotide primers, detection probes, and/or additional reagents into a plurality of reaction volumes. In some embodiments, the present disclosure is enabled by the use of digital PCR (or other partition-based assay with multiple PCR sub-reactions). In one example of dPCR, a single sample containing a nucleic acid target sequence, an amplification oligomer, a detection oligonucleotide, dNTPs, a thermostable DNA polymerase, and other PCR reagents may be partitioned into approximately 20,000 evenly sized reaction volumes.

A reaction volumes may be a droplet (e.g., a droplet in an emulsion). A reaction volume may be a microdroplet. A reaction volume may be a well. A reaction volume may be a microwell. Partitioning may be performed using a microfluidic device. In some cases, partitioning is performed using a droplet generator. Partitioning may include dividing a sample or sample into water-in-oil droplets.

Generally, each reaction volume may receive a single template of the nucleic acid target sequence. However, statistically, some reaction volumes may receive more than one copy of a nucleic acid target template, while other reaction volumes may not receive any target template. Accordingly, a droplet may include a single nucleic acid. A droplet may include one or more nucleic acids. A droplet may include no nucleic acids. Each droplet of a plurality of droplets may generate a signal. A plurality of signals may include the signal(s) generated from each of a plurality of droplets including a subset of a sample.

In some aspects, following partitioning, each reaction volume may be subject to end-point PCR. In embodiments, reaction volumes emitting a fluorescent signal are marked “positive” and scored as “1,” whereas partitions without detectable fluorescence are deemed “negative” and scored as “0.” The underlining theory of dPCR is that the number of positive reactions is directly proportional to the total number of template nucleic acid present in the sample—thereby enabling absolute quantification.

In some embodiments, the present disclosure provides methods, systems, and compositions for multiplex quantification using digital assays, wherein individual reaction volumes need not be classified to quantify targets present in a reaction. The disclosed methods may be useful in identifying or detecting genetic abnormalities from a subject, for example, fetal aneuploidy (e.g., trisomy 21, trisomy 18, etc.).

Amplification

In embodiments, the disclosed methods may include nucleic acid amplification. Amplification conditions may include thermal cycling conditions, including temperature and length in time of each thermal cycle. The use of particular amplification conditions may serve to modify the signal intensity of each signal, thereby enabling each signal to correspond to a unique combination of nucleic acid targets. Amplification may include using enzymes such to produce additional copies of a nucleic. The amplification reaction may include using oligonucleotide primers (“primers” or “amplification oligomers” as described elsewhere herein). The oligonucleotide primers may use specific sequences to amplify a specific sequence. The oligonucleotide primers may amplify a specific sequence by hybridizing to a sequence upstream and downstream of the primers and result in amplifying the sequence inclusively between the upstream and downstream primer. The amplification reaction may include the use of nucleotide tri-phosphate reagents. The nucleotide tri-phosphate reagents may include using deoxyribo-nucleotide tri-phosphate (dNTPs). The nucleotide tri-phosphate reagents may be used as precursors to the amplified nucleic acids. The amplification reaction may include using oligonucleotide probes as described elsewhere herein. The amplification reaction may include using enzymes. Non-limiting examples of enzymes include thermostable enzymes, DNA polymerases, RNA polymerases, and reverse transcriptases. The amplification reaction may include generating nucleic acid molecules of a different nucleotide types. For example, a target nucleic acid may include DNA and an RNA molecule may be generated. In another example, an RNA molecule may be subjected to an amplification reaction and a cDNA molecule may be generated.

Thermal Cycling

Methods of the present disclosure may include thermal cycling. Thermal cycling may include one or more thermal cycles. Thermally cycling may be performed under reaction conditions appropriate to amplify a template nucleic acid with PCR. Amplification of a template nucleic acid may require binding or annealing of oligonucleotide primer(s) to the template nucleic acid. Appropriate reaction conditions may include appropriate temperature conditions, appropriate buffer conditions, and the presence of appropriate reagents. Appropriate temperature conditions may, in some cases, be such that each thermal cycle is performed at a desired annealing temperature. A desired annealing temperature may be sufficient for annealing of an oligonucleotide probe(s) to a nucleic acid target. Appropriate buffer conditions may, in some cases, be such that the appropriate salts are present in a buffer used during thermal cycling. Appropriate salts may include magnesium salts, potassium salts, ammonium salts. Appropriate buffer conditions may be such that the appropriate salts are present in appropriate concentrations. Appropriate reagents for amplification of each member of a plurality of nucleic acid targets with PCR may include deoxyribonucleotide triphosphates (dNTPs). dNTPs may include natural or non-natural dNTPs including, for example, dATP, dCTP, dGTP, dTTP, dUTP, and variants thereof.

In various aspects, primer extension reactions may be utilized to generate amplified product. Primer extension reactions generally include a cycle of incubating a reaction mixture at a denaturation temperature for a denaturation duration and incubating a reaction mixture at an elongation temperature for an elongation duration. In any of the various aspects, multiple cycles of a primer extension reaction can be conducted. Any suitable number of cycles may be conducted. For example, the number of cycles conducted may be between about 5 cycles to about 100 cycles. The number of cycles conducted may depend upon, for example, the number of cycles (e.g., cycle threshold value (Ct)) used to obtain a detectable amplified product (e.g., a detectable amount of amplified DNA product that is indicative of the presence of a target DNA in a nucleic acid sample).

The time for which an amplification reaction yields a detectable amount of amplified nucleic acid may vary depending upon the nucleic acid sample, the sequence of the target nucleic acid, the sequence of the primers, the particular nucleic acid amplification reactions conducted, and the particular number of cycles of the amplification, the temperature of the reaction, the pH of the reaction. For example, amplification of a target nucleic acid may yield a detectable amount of product indicative to the presence of the target nucleic acid at time period between about 5 minutes to about 120 minutes.

Nucleic Acid Targets

In embodiments, a nucleic acid target of the present disclosure may be derived from a biological sample. A biological sample may be a sample derived from a subject. A biological sample may include any number of macromolecules, for example, cellular macromolecules. A biological sample may be derived from another sample. A biological sample may be a tissue sample, such as a biopsy, core biopsy, needle aspirate, or fine needle aspirate. A biological sample may be a fluid sample, such as a blood sample, urine sample, or saliva sample. A biological sample may be a skin sample. A biological sample may be a cheek swab. A biological sample may be a plasma or serum sample. A biological sample may include one or more cells. A biological sample may be, for example, blood, plasma, serum, urine, saliva, mucosal excretions, sputum, stool or tears.

In some embodiments, a nucleic acid target may be derived from one or more cells. A nucleic acid target may include deoxyribonucleic acid (DNA). DNA may be any kind of DNA, including genomic DNA. A nucleic acid target may be viral DNA. A nucleic acid target may include ribonucleic acid (RNA). RNA may be any kind of RNA, including messenger RNA, transfer RNA, ribosomal RNA, and microRNA. RNA may be viral RNA.

In embodiments, nucleic acid targets may include one or more members. A member may be any region of a nucleic acid target. A member may be of any length. A member may be, for example, up to about 1 to about 100000 nucleotides, or more. In some instances, a member may be a gene. A nucleic acid target may include a gene whose detection may be useful in diagnosing one or more diseases. A gene may be a viral gene or bacterial gene whose detection may be useful in identifying the presence or absence of a pathogen in a subject. In some cases, the methods of the present disclosure are useful in detecting the presence or absence or one or more infectious agents (e.g., viruses, bacteria, fungi) in a subject. The nucleic acid targets may be a human gene. In some cases, the methods of the present disclosure are useful in identifying or detecting genetic abnormalities from a subject, for example, fetal aneuploidy (e.g., trisomy 21, trisomy 18, etc.).

In embodiments, a nucleic acid target may be cell-free nucleic acid. Cell-free nucleic acid may be, for example, cell-free tumor DNA, cell-free fetal DNA, cell-free RNA, etc. In some cases, a nucleic acid derived from a cell-free sample may be derived from plasma of a subject. For example, nucleic acid (e.g., maternal nucleic acid and fetal nucleic acid) may be derived from a plasma sample of a pregnant female. A nucleic acid may be a fetal nucleic acid. A nucleic acid may be a maternal nucleic acid. A nucleic acid sequence may correspond to a region of a nucleic acid potentially associated with an aneuploidy (e.g., a nucleic acid sequence of a fetal nucleic acid may be associated with a fetal aneuploidy). In some cases, the methods of the present disclosure are useful in identifying and estimating a fetal fraction in a sample. In some cases, the methods of the present disclosure are useful in identifying the presence or absence of a fetal aneuploidy in a sample. In some cases, the methods of the present disclosure are useful in detecting the relative amount of a fetal nucleic acid in a cell-free nucleic acid sample from a subject, thereby diagnosing the fetus for one or more genetic abnormalities (e.g., fetal aneuploidy).

In some aspects, one or more nucleic acid molecules analyzed by methods of the present disclosure may correspond to a chromosome. A nucleic acid sequence may be a region of a chromosome associated with a fetal aneuploidy. Chromosomes associated with fetal aneuploidy may include, for example, chromosome 21 (e.g., associated with trisomy 21), chromosome 18 (e.g., associated with trisomy 18), chromosome 13 (e.g., associated with trisomy 13), and an X chromosome (e.g., associated with sex chromosome aneuploidies).

In some embodiments, the nucleic acid target may be a region of the human genome that indicates a predisposition for a particular disease. Markers for a predisposition for a particular disease may be detected by specific mutations or SNPs that are associated with higher infections. For example, a particular mutation or SNP of in a subject may be associated with an increased risk of infection of a particular pathogen. The detection of both a pathogenic nucleic acid sequence and the presence of a SNP in the subject's genome may indicate the subject is at a high risk.

Nucleic acid targets may be of various concentrations in the reaction. The nucleic acid sample may be diluted or concentrated to achieve different concentrations of nucleic acids. The concentration of the nucleic acids in the nucleic acid sample may include between 0.1 nanograms per microliter (ng/μL) to about 10000 ng/μL.

Sample

As described above, a nucleic acid target of the present disclosure may be derived from a biological sample. A biological sample may be a sample derived from an organism. A biological sample may include any number of macromolecules, for example, cellular macromolecules. A biological sample may be, for example, blood, plasma, serum, urine, saliva, mucosal excretions, sputum, stool or tears. A biological sample may be a fluid sample. A fluid sample may be blood or plasma. A nucleic acid target may be a nucleic acid from a pathogen (e.g., virus, bacteria, etc.). A nucleic acid target may be a nucleic acid suspected of having one or more mutations. A biological sample may be derived from another sample. A biological sample may be a tissue sample, such as a biopsy, core biopsy, needle aspirate, or fine needle aspirate. A biological sample may be a fluid sample, such as a blood sample, urine sample, or saliva sample. A biological sample may be a skin sample. A biological sample may be a cheek swab. A biological sample may be a plasma or serum sample. A biological sample may include one or more cells. A biological sample may be a cell-free sample (e.g., cell-free RNA, cell-free DNA, etc.). A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

Sample Processing

In embodiments, a sample may be processed concurrently with, prior to, or subsequent to the methods of the present disclosure. A sample may be processed to purify or enrich for nucleic acids (e.g., to purify nucleic acids from a plasma sample). A sample of nucleic acids may be processed to purity or enrich for nucleic acid of interest. A sample of nucleic acids may be processed to enrich for fetal nucleic acid. A sample of nucleic acids may be processed to enrich for nucleic acid fragments smaller than a given size.

In some embodiments, a sample may be enriched for nucleic acid of interest (e.g., fetal nucleic acid) by various methods including, for example, by size exclusion filtration, sequence-specific enrichment (e.g., via use of capture sequences), epigenetic-specific enrichment (e.g., via use of methylation-sensitive restriction enzymes (MSREs)). Enrichment may include isolation of nucleic acid of interest and/or depletion of nucleic acid that is not of interest.

In some embodiments, a sample is not processed to purify or enrich for nucleic acid of interest prior to performing methods of the present disclosure (e.g., amplification of nucleic acids from a sample). In some examples, a sample is not processed to enrich for fetal nucleic acid prior to mixing a sample with oligonucleotide primers and oligonucleotide probes, as described elsewhere herein. The disclosed methods may be capable of, for example, identifying fetal fraction and/or identifying a fetal aneuploidy regardless of whether a sample has been purified or enriched for fetal nucleic acid.

Nucleic Acid Enzymes

In embodiments, mixtures and compositions of the present disclosure may include one or more nucleic acid enzymes. A nucleic acid enzyme may have exonuclease activity. A nucleic acid enzyme may have endonuclease activity. A nucleic acid enzyme may have RNase activity. A nucleic acid enzyme may be capable of degrading a nucleic acid including one or more ribonucleotide bases. A nucleic acid enzyme may be, for example, RNase H or RNase III. An RNase III may be, for example, Dicer. A nucleic acid may be an endonuclease I such as, for example, a T7 endonuclease I. A nucleic acid enzyme may be capable of degrading a nucleic acid including a non-natural nucleotide. A nucleic acid enzyme may be an endonuclease V such as, for example, an E. coli endonuclease V.

A nucleic acid enzyme may be a polymerase (e.g., a DNA polymerase). A DNA polymerase may be used. Any suitable DNA polymerase may be used, including commercially available DNA polymerases. A DNA polymerase generally refers to an enzyme that is capable of incorporating nucleotides to a strand of DNA in a template bound fashion. A polymerase may include Taq polymerase or a variant thereof. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C.-95° C. for 2 minutes to 10 minutes may be required, which may change the thermal profile based on different polymerases.

In some embodiments, a nucleic acid enzyme may be capable, under appropriate conditions, of degrading an oligonucleotide probe. For example, a nucleic acid enzyme may be a polymerase and include exo activity and degrade a probe resulting in a detectable signal. A nucleic acid enzyme may be capable, under appropriate conditions, of releasing a quencher from an oligonucleotide probe.

Reactions

In embodiments, a reaction may include contacting nucleic acid targets with one or more oligonucleotide probes (also known herein as a detection probe). A reaction may include the use of intercalating dyes, thereby removing the need for probes. A reaction may include contacting a sample solution volume (e.g., a droplet, well, tube, etc.) with a plurality of oligonucleotide probes, each corresponding to one of a plurality of nucleic acid targets, to generate a plurality of signals generated from the plurality of oligonucleotide probes. A reaction may include polymerase chain reaction (PCR). A reaction may be a digital PCR (dPCR) reaction.

Oligonucleotide Primers

In various aspects disclosed elsewhere herein, oligonucleotide primers are used. An oligonucleotide primer (or just “primer” or “amplification oligomer”) of the present disclosure may be a deoxyribonucleic acid. An oligonucleotide primer may be a ribonucleic acid. An oligonucleotide primer may include one or more non-natural nucleotides. A non-natural nucleotide may be, for example, deoxyinosine.

An oligonucleotide primer may be a forward primer. An oligonucleotide primer may be a reverse primer. An oligonucleotide primer may be between about 5 and about 50 nucleotides in length. An oligonucleotide primer may range between 5 and 50 base pairs in length. In some examples, an oligonucleotide primer may exceed 50 base pairs in length. In some examples, the oligonucleotide primers may be between 5-50 base pairs in length. In embodiments, the oligonucleotide primers may be about 5, 10, 11, 12, 13, 14, 15, 16, 17, 18, 19, 20, 25, 30, 35, 40, 45, or about 50 base pairs in length.

A set of oligonucleotide primers may include paired oligonucleotide primers. Paired oligonucleotide primers may include a forward oligonucleotide primer and a reverse oligonucleotide primer. A forward oligonucleotide primer may be configured to hybridize to a first region (e.g., a 3′ end) of a nucleic acid sequence, and a reverse oligonucleotide primer may be configured to hybridize to a second region (e.g., a 5′ end) of the nucleic acid sequence, thereby being configured to amplify the nucleic acid sequence under conditions sufficient for nucleic acid amplification. Different sets of oligonucleotide primers may be configured to amplify different nucleic acid target sequences. For example, a first set of oligonucleotide primers may be configured to amplify a first nucleic acid sequence of a given length, and a second set of oligonucleotide primers may be configured to amplify a second nucleic acid sequence of shorter length than the first nucleic acid sequence. In another example, a first set of oligonucleotide primers may be configured to amplify a first nucleic acid sequence of a given length, and a second set of oligonucleotide primers may be configured to amplify a second nucleic acid sequence of longer length than the first nucleic acid sequence.

A mixture may include forward oligonucleotide primers. Forward oligonucleotide primers may include a deoxyribonucleic acid and/or a ribonucleic acid. Forward oligonucleotide primers may be between about 5 and about 50 nucleotides in length. In some example, a forward oligonucleotide primers may exceed 50 nucleotides in length. In some examples, the forward oligonucleotide primer may be between 5-50 base pairs in length. In some embodiments, the base pairs may be about 5, 10, 11, 12, 13, 14, 15, 16, 17, 18, 19, 20, 25, 30, 35, 40, 45, or 50 base pairs in length.

A set of oligonucleotide primers (e.g., a forward primer and a reverse primer) may be configured to amplify a nucleic acid sequence of a given length (e.g., may hybridize to regions of a nucleic acid sequence a given distance apart). A pair of oligonucleotide primers may be configured to amplify a nucleic acid sequence of a length of between 50 and 300 base pairs, or more. In some examples, the pair of oligonucleotide primers may be configured to amplify a nucleic acid sequence by 50, 75, 100, 125, 150, 175, 200, 225, 250, 275, or 300 base pairs.

In some embodiments, a mixture may include one or more synthetic (or otherwise generated to be different from the target of interest) primers for PCR reactions.

In some embodiments, a mixture may be subjected to conditions sufficient to anneal an oligonucleotide primer to a nucleic acid molecule. In some aspects, a mixture may be subjected to conditions sufficient to anneal a plurality of oligonucleotide primers to a nucleic acid molecule. In some aspects, a mixture may be subjected to conditions sufficient to anneal a plurality of oligonucleotide primers to a plurality of nucleic acid targets. The mixture may be subjected to conditions which are sufficient to denature nucleic acid molecules. Subjecting a mixture to conditions sufficient to anneal an oligonucleotide primer to a nucleic acid target may include thermally cycling the mixture under reaction conditions appropriate to amplify the nucleic acid target(s) with, for example, polymerase chain reaction (PCR).

Conditions may be such that an oligonucleotide primer pair (e.g., forward oligonucleotide primer and reverse oligonucleotide primer) are degraded by a nucleic acid enzyme. An oligonucleotide primer pair may be degraded by the exonuclease activity of a nucleic acid enzyme. An oligonucleotide primer pair may be degraded by the RNase activity of a nucleic acid enzyme. Degradation of the oligonucleotide primer pair may result in release of the oligonucleotide primer. Once released, the oligonucleotide primer pair may bind or anneal to a template nucleic acid.

Detection Probes or Oligonucleotide Probes

In embodiments, oligonucleotide probes may be used. In some aspects, the oligonucleotide probe may also be referenced herein as a “detection probe” or “probe”. An oligonucleotide probe may be a nucleic acid (e.g., DNA, RNA, etc.). An oligonucleotide probe may include a region complementary to a region of a nucleic acid target. The concentration of an oligonucleotide probe may be such that it is in excess relative to other components in a sample.

An oligonucleotide probe may include a non-target-hybridizing sequence. A non-target-hybridizing sequence may be a sequence which is not complementary to any region of a nucleic acid target sequence. An oligonucleotide probe of a non-target-hybridizing sequence may be a hairpin detection probe. An oligonucleotide probe of a non-target-hybridizing sequence may be a molecular beacon probe. Examples of molecular beacon probes are generally known in the art. An oligonucleotide probe of a non-target-hybridizing sequence may be a molecular torch.

A sample may include oligonucleotide probes. The oligonucleotide probes may be the same or may be different. An oligonucleotide probe may range between 5 and 30 nucleotides in length. In some examples, the oligonucleotide probe may exceed 30 nucleotides in length. In some examples, a mixture includes a first oligonucleotide probe and one or more additional oligonucleotide probes. An oligonucleotide probe may be a nucleic acid (e.g., DNA, RNA, etc.). An oligonucleotide probe may range between 2 and 50 nucleotides in length, or more. In some examples, an oligonucleotide probe may be 2, 3, 4, 5, 6, 7, 8, 9, 10, 20, 30, 40, or 50 nucleotides in length.

In some examples, 2, 3, 4, 5, 6, 7, 8, 9, 10, 11, 12, 13, 14, 15, 20, or more different oligonucleotide probes may be used. Each oligonucleotide probe may correspond to (e.g., capable of binding to) a given region of a nucleic acid target (e.g., a chromosome) in a sample. In one example, a first oligonucleotide probe is specific for a first region of a first nucleic acid target, a second oligonucleotide probe is specific for a second region of the first nucleic acid target, and a third oligonucleotide probe is specific for a third region of the first nucleic acid target. Each oligonucleotide probe may include a signal tag with about equal emission wavelengths. In some examples, each oligonucleotide probe includes an identical fluorophore. In some examples, each oligonucleotide probe includes a different fluorophore. In some case, each fluorophore is capable of being detected in a single optical channel. In some examples, a fluorophore may be detected in multiple channels. In some examples, an oligonucleotide probe may have similar or the same detectable agent or fluorophore as another oligonucleotide probe in the sample. In some examples, an oligonucleotide probe may have a different detectable agent or fluorophore as compared to another oligonucleotide probe in the sample. In some cases, an oligonucleotide probe may have similar sequence or be capable or binding a similar sequence as another oligonucleotide probe in the sample. In some examples, an oligonucleotide probe may have a different sequence or be capable of binding a different sequence as compared to another oligonucleotide probe in the sample.

A probe may correspond to a region of a nucleic acid target. For example, a probe may have complementarity and/or homology to a region of a nucleic acid target. A probe may include a region which is complementary or homologous to a region of a nucleic acid target. A probe corresponding to a region of a nucleic acid target may be capable of binding to the region of the nucleic acid target under appropriate conditions (e.g., temperature conditions, buffer conditions. etc.). For example, a probe may be capable of binding to a region of a nucleic acid target under conditions appropriate for polymerase chain reaction. A probe may correspond to an oligonucleotide which corresponds to a nucleic acid target. For example, an oligonucleotide may be a primer with a region complementary to a nucleic acid target and a region complementary to a probe.

A probe may be provided at a specific concentration. In some examples, a second nucleic acid probe is provided at a concentration of between 2X and 8X. In some examples, a second nucleic acid probe is provided at a concentration of about 2X, about 3X, about 4X, about 5X, about 6X, about 7X, about 8X, or more. In some examples, a second nucleic acid probe is provided at a concentration of about 8X, about 7X, about 6X, about 5X, about 4X, about 3X, or about 2X. In some cases, a second nucleic acid probe is provided at a concentration of about 2X, about 3X, about 4X, about 5X, about 6X, about 7X, or about 8X. X may be a concentration of a nucleic acid probe provided in the disclosed methods. In some examples, X ranges from 50 nM to 500 nM, or greater. In some examples, X may be about 50 nM, 100 nM, 150 nM, 200 nM, 250 nM, 300 nM, 350 nM, 400 nM, 450 nM, 500 nM. X may be any concentration of a nucleic acid probe.

A probe may include a fluorophore. A fluorophore may be selected from any number of fluorophores. A fluorophore may be selected from three, four, five, six, seven, eight, nine, or ten fluorophores, or more. One or more oligonucleotide probes used in a single reaction may include the same fluorophore. In some cases, all oligonucleotide probes used in a single reaction include the same fluorophore. Each probe may, when excited and contacted with its corresponding nucleic acid target, generate a signal. A signal may be a fluorescent signal. A plurality of signals may be generated from one or more probes.

An oligonucleotide probe may have less than 50%, 40%, 30%, 20%, 10%, 5%, or 1% complementarity to any member of a plurality of nucleic acid targets. An oligonucleotide probe may have no complementarity to any member of the plurality of nucleic acid targets.

An oligonucleotide probe may include a detectable label. A detectable label may be a chemiluminescent label. A detectable label may include a fluorescent label. A detectable label may include a fluorophore. Non-limiting examples of fluorescent molecules that may be used include Alexa Fluor 350, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750, Cy3, Cy5, Texas Red, Fluorescein (FITC), 6-FAM, 5-FAM, HEX, JOE, TAMRA, ROX, BODIPY FL, Pacific Blue, Pacific Green, Coumarin, Oregon Green, Pacific Orange, Trimethylrhodamine (TRITC), DAPI, APC, Cyan Fluorescent Protein (CFP), Green Fluorescent Protein (GFP), Red Fluorescent Protein (RFP), Phycoerythin (PE), quantum dots (for example, Qdot 525, Qdot 565, Qdot 605, Qdot 705, Qdot 800), or derivatives thereof. An oligonucleotide probe may further include one or more quenchers. A quencher may inhibit signal generation from a fluorophore. A quencher may be, for example, TAMRA, BHQ-1, BHQ-2, or Dabcy. A quencher may be BHQ-1. A quencher may be BHQ-2.

Signal Generation

In embodiments, thermal cycling may be performed such that one or more oligonucleotide probes are degraded by a nucleic acid enzyme. An oligonucleotide probe may be degraded by the exonuclease activity of a nucleic acid enzyme. An oligonucleotide probe may generate a signal upon degradation. In some cases, an oligonucleotide probe may generate a signal only if at least one member of a plurality of nucleic acid targets is present in a sample.

A reaction may generate one or more signals. A reaction may generate a cumulative intensity signal including a sum aggregate of multiple signals. A signal may be a chemiluminescent signal. A signal may be a fluorescent signal. A signal may be generated by an oligonucleotide probe. A sum signal may be generated by at least one oligonucleotide probe. For example, excitation of a hybridization probe including a luminescent signal tag may generate a signal. A signal may be generated by a fluorophore. A fluorophore may generate a signal upon release from a hybridization probe. A reaction may include excitation of a fluorophore. A reaction may include signal detection. A reaction may include detecting emission from a fluorophore.

A signal may be a fluorescent signal. A signal may correspond to a fluorescence intensity level. Each signal measured in the methods of the present disclosure may have a distinct fluorescence intensity value, thereby corresponding to the presence of a unique combination of nucleic acid targets. A signal may be generated by one or more oligonucleotide probes. The number of signals generated in an assay may correspond to the number of oligonucleotide probes and nucleic acid targets present.

N may be a number of signals detected in a single optical channel in an assay of the present disclosure. N may range between 2 and 50, or more. In some examples, N may be 2, 3, 4, 5, 6, 7, 8, 9, 10, 11, 12, 13, 14, 15, 20, 25, 30, 40, or 50.

In some cases, a sum signal may be detected in a single optical channel. In some cases, a sum signal may be detected in at least one optical channel, thereby significantly increasing the number of nucleic acid targets that can be measured in a single reaction (e.g., digital PCR reaction). In some cases, two sets of signals are detected in a single reaction. Each set of signals detected in a reaction may includes the same number of signals, or different numbers of signals.

In some cases, a signal may be generated simultaneous with hybridization of an oligonucleotide probe to a region of a nucleic acid. For example, an oligonucleotide probe (e.g., a molecular beacon probe or molecular torch) may generate a signal (e.g., a fluorescent signal) following hybridization to a nucleic acid.

In cases where an oligonucleotide probe includes a signal tag, the oligonucleotide probe may be degraded when bound to a region of an oligonucleotide primer, thereby generating a signal. For example, an oligonucleotide probe (e.g., a TaqMan® probe) may generate a signal following hybridization of the oligonucleotide probe to a nucleic acid and subsequent degradation by a polymerase (e.g., during amplification, such as PCR amplification). An oligonucleotide probe may be degraded by the exonuclease activity of a nucleic acid enzyme.

An oligonucleotide probe may include a quencher and a fluorophore, such that the quencher is released upon degradation of an oligonucleotide probe, thereby generating a fluorescent signal. Thermal cycling may be used to generate one or more signals. Thermal cycling may generate at least one, two, three, four, five, six, seven, eight, nine, ten signals, or more. Thermal cycling may generate at most ten, nine, eight, seven, six, five, four, three, two or one signal.

Multiple signals may be of the same type or of different types. Signals of different types may be fluorescent signals with different fluorescent wavelengths. Signals of different types may be generated by detectable labels that include different fluorophores. Signals of the same type may be of different intensities (e.g., different intensities of the same fluorescent wavelength). Signals of the same type may be signals detectable in the same color channel. Signals of the same type may be generated by detectable labels that may include the same fluorophore. Detectable labels that may include the same fluorophore may generate different signals by nature of being at different concentrations, thereby generating different intensities of the same signal type.

Although fluorescent probes have been used to illustrate this principle, the disclosed methods are equally applicable to any other method providing a quantifiable signal, including an electrochemical signal, chemiluminescent signals, magnetic particles, and electrets structures exhibiting a permanent dipole.

In certain portions of this disclosure, the signal may be a fluorescent signal. For example, like fluorescent signals, any of the electromagnetic signals described above may also be characterized in terms of a wavelength, whereby the wavelength of a fluorescent signal may also be described in terms of color. The color may be determined based on measuring intensity at a particular wavelength or range of wavelengths, for example by determining a distribution of fluorescent intensity at different wavelengths and/or by utilizing a band pass filter to determine the fluorescence intensity within a particular range of wavelengths.

The signal is described is not meant to be limiting, and one of ordinary skill in the art will readily recognize that the principles applicable to the measurement of a fluorescent signal are also applicable to other signals. For example, the methods presented in this disclosure may also utilize the measurement of a signal in at least two dimensions (e.g., color and intensity). In some cases, a quantifiable signal has both a frequency (wavelength) and an amplitude (intensity). A signal may be an electromagnetic signal. An electromagnetic signal may be a sound, a radio signal, a microwave signal, an infrared signal, a visible light signal, an ultraviolet light signal, an x-ray signal, or a gamma-ray signal. The wavelength of a fluorescent signal may also be described in terms of color. The color may be determined based on measuring intensity at a particular wavelength or range of wavelengths, for example by determining a distribution of fluorescent intensity at different wavelengths and/or by utilizing a band pass filter to determine the fluorescence intensity within a particular range of wavelengths. Intensity may be measured with a photodetector. A range of wavelengths may be referred to as a “channel,” “color channel,” or “optical channel.”

In some cases, the signal intensity increases with each thermal cycle. The signal intensity may increase in a sigmoidal fashion. The presence of a signal may be correlated to the presence of at least one member of a plurality of target nucleic acids. Correlating the presence of a signal to the presence of at least one member of a plurality of target nucleic acids may include establishing a signal intensity threshold. A signal intensity threshold may be different for different signals. Correlating the presence of a signal to the presence of at least one member of a plurality of target nucleic acids may include determining whether the intensity of a signal increases beyond a signal intensity threshold. In some examples, the presence of a signal may be correlated with the presence of at least one of all members of a plurality of target nucleic acids. In other examples, the presence of a first signal may be correlated with the presence of at least one of a first subset of members of a plurality of target nucleic acids, and the presence of a second signal may be correlated with the presence of at least one of a second subset of members of a plurality of target nucleic acids.

The presence of a signal may be correlated to the presence of a nucleic acid target. The presence of between 1 and 10 signals may be correlated with the presence of between 1 and 10 nucleic acid targets. The absence of a signal may be correlated with the absence of corresponding nucleic acid targets. The absence of 1, 2, 3, 4, 5, 6, 7, 8, 9, 10, or more signals may be correlated with the absence of each of 1, 2, 3, 4, 5, 6, 7, 8, 9, 10, or more nucleic acid target molecules.

Detection

In embodiments, the presence or absence of one or more signals may be detected. One signal may be detected, or multiple signals may be detected. Multiple signals may be detected simultaneously. Alternatively, multiple signals may be detected sequentially. The presence of a signal may be correlated to the presence of a nucleic acid target. The presence of one, two, three, four, five, six, seven, eight, nine, ten, or more signals may be correlated with the presence of one, two, three, four, five, six, seven, eight, nine, ten, or more nucleic acid targets. The absence of a signal may be correlated with the absence of corresponding nucleic acid targets. The absence of one, two, three, four, five, six, seven, eight, nine, ten, or more signals may be correlated with the absence of each of one two, three, four, five, six, seven, eight, nine, ten, or more nucleic acid target molecules.

Detecting Multiple Nucleic Acid Targets

Various methods described herein may be used to differentially quantify nucleic acid sequences in a biological sample and to determine whether the nucleic acid sequences originate from a single source or from two or more sources. In embodiments, differential quantification may be performed by using differentially methylated loci, fragment size distribution, and/or single nucleotide polymorphisms (SNPs).

Technology

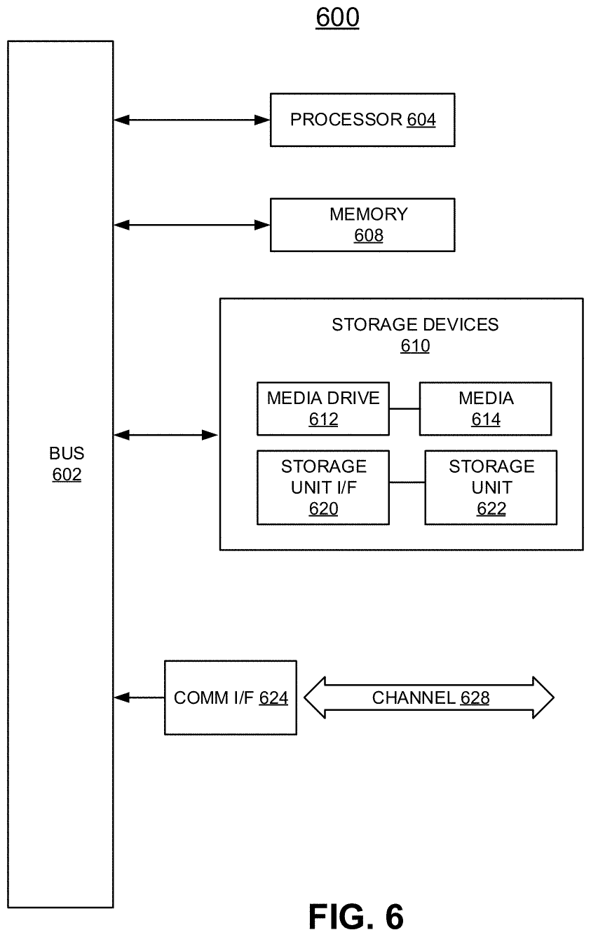

FIG. 6 illustrates example computing component 600. Computing component 600 may be used to implement various features and/or functionality of embodiments of the systems, devices, and methods disclosed herein. With regard to the above-described embodiments set forth herein in the context of methods and systems described with reference to FIGS. 1 through 5, one of skill in the art will appreciate additional variations and details regarding the functionality of these embodiments that may be carried out by computing component 600. In this connection, it will also be appreciated by one of skill in the art upon studying the present disclosure that features and aspects of the various embodiments (e.g., systems) described herein may be implemented with respected to other embodiments (e.g., methods) described herein without departing from the spirit of the disclosure.

As used herein, the term component may describe a given unit of functionality that may be performed in accordance with one or more embodiments of the present application. As used herein, a component references a module, and/or may be implemented utilizing any form of hardware, software, or a combination thereof. For example, one or more processors, controllers, ASICs, PLAs, PALs, CPLDs, FPGAs, logical components, software routines or other mechanisms may be implemented to make up a component. In embodiment, the various components described herein may be implemented as discrete components or the functions and features described may be shared in part or in total among one or more components. In other words, as would be apparent to one of ordinary skill in the art after reading this description, the various features and functionality described herein may be implemented in any given application and may be implemented in one or more separate or shared components in various combinations and permutations. Even though various features or elements of functionality may be individually described or claimed as separate components, one of ordinary skill in the art will understand upon studying the present disclosure that these features and functionality may be shared among one or more common software and hardware elements, and such description shall not require or imply that separate hardware or software components are used to implement such features or functionality.

Where components of the application are implemented in whole or in part using software, in one embodiment, these software elements can be implemented to operate with a computing or processing component capable of carrying out the functionality described with respect thereto. One such example computing component is shown in FIG. 6. Various embodiments are described in terms of this example-computing component 600. After reading this description, it will become apparent to a person skilled in the relevant art how to implement the application using other computing components or architectures.

Referring now to FIG. 6, computing component 600 may represent, for example, computing or processing capabilities found within a self-adjusting display, desktop, laptop, notebook, and tablet computers; hand-held computing devices (tablets, PDA's, smart phones, cell phones, palmtops, etc.); workstations or other devices with displays; servers; or any other type of special-purpose or general-purpose computing devices as may be desirable or appropriate for a given application or environment. Computing component 600 might also represent computing capabilities embedded within or otherwise available to a given device. For example, a computing component might be found in other electronic devices such as, for example navigation systems, portable computing devices, and other electronic devices that might include some form of processing capability.

Computing component 600 might include, for example, one or more processors, controllers, control components, or other processing devices, such as a processor 604. Processor 604 might be implemented using a general-purpose or special-purpose processing engine such as, for example, a microprocessor, controller, or other control logic. In the illustrated example, processor 604 is connected to a bus 602, although any communication medium can be used to facilitate interaction with other components of computing component 600 or to communicate externally.

Computing component 600 might also include one or more memory components, simply referred to herein as main memory 608. For example, preferably random access memory (RAM) or other static or dynamic memory, might be used for storing information and instructions to be executed by processor 604. Main memory 608 might also be used for storing temporary variables or other intermediate information during execution of instructions to be executed by processor 604. Computing component 600 might likewise include a read only memory (“ROM”) or other static storage device coupled to bus 602 for storing static information and instructions for processor 604.

The computing component 600 might also include one or more various forms of information storage mechanism 610, which might include, for example, a media drive 612 and a storage unit interface 620. The media drive 612 might include a drive or other mechanism to support fixed or removable storage media 614. For example, a hard disk drive, a solid state drive, a magnetic tape drive, an optical disk drive, a compact disc (CD) or digital video disc (DVD) drive (R or RW), or other removable or fixed media drive might be provided. Accordingly, storage media 614 might include, for example, a hard disk, flash drive, an integrated circuit assembly, USB, magnetic tape, cartridge, optical disk, a CD or DVD, or other fixed or removable medium that is read by, written to or accessed by media drive 612. As these examples illustrate, the storage media 614 can include a computer usable storage medium having stored therein computer software or data.

In alternative embodiments, information storage mechanism 610 might include other similar instrumentalities for allowing computer programs or other instructions or data to be loaded into computing component 600. Such instrumentalities might include, for example, a fixed or removable storage unit 622 and an interface 620. Examples of such storage units 622 and interfaces 620 can include a program cartridge and cartridge interface, a removable memory (for example, a flash memory or other removable memory component) and memory slot, a PCMCIA slot and card, and other fixed or removable storage units 622 and interfaces 620 that allow software and data to be transferred from the storage unit 622 to computing component 600.

Computing component 600 might also include a communications interface 624. Communications interface 624 might be used to allow software and data to be transferred between computing component 600 and external devices. Examples of communications interface 624 might include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 624 might typically be carried on signals, which can be electronic, electromagnetic (which includes optical) or other signals capable of being exchanged by a given communications interface 624. These signals might be provided to communications interface 624 via a channel 628. This channel 628 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media such as, for example, memory 608, storage unit 620, media 614, and channel 628. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing component 1000 to perform features or functions of the present application as discussed herein.

Kits

The present disclosure also provides kits for multiplex analysis. Kits may be useful in, for example, analyzing nucleic acid size distribution (e.g., fetal fraction), determining the methylation status of a sample, and/or determining the number of SNPs of a sample. In some embodiments, the kit may include amplification reagents including, for example, oligonucleotide primers, oligonucleotide probes, dNTPs, a nucleic acid enzyme (e.g., a polymerase), and salts (e.g., Ca2+, Mg2+, etc.). In some cases, the reagents may be used in a quantitative Polymerase Chain Reaction (qPCR), whereby a plurality of signals may be generated. In some cases, the reagents may be used in a digital Polymerase Chain Reaction (dPCR), whereby a plurality of signals may be generated. Kits may include one or more oligonucleotide probes. Oligonucleotide probes may be lyophilized. Different oligonucleotide probes may be present at different concentrations in a kit. Oligonucleotide probes may include a fluorophore and/or one or more quenchers.

In embodiments, a kit may include one or more pairs of oligonucleotide primers as described herein. Paired oligonucleotide primers may include a forward oligonucleotide primer and a reverse oligonucleotide primer. A pair of oligonucleotide primers may be configured to amplify a nucleic acid sequence corresponding to particular targets. Different pairs of oligonucleotide primers may be configured to amplify nucleic acid sequences. Oligonucleotide primers configured to amplify nucleic acid molecules may be used in performing the disclosed methods. In some cases, all of the oligonucleotide primers in a kit are lyophilized.

In embodiments, a kit may include one or more nucleic acid enzymes. A nucleic acid enzyme may be a nucleic acid polymerase. A nucleic acid polymerase may be a deoxyribonucleic acid polymerase (DNase). A DNase may be a Taq polymerase or variant thereof. A nucleic acid enzyme may be a ribonucleic acid polymerase (RNase). An RNase may be an RNase III. An RNase III may be Dicer. The nucleic acid enzyme may be an endonuclease. An endonuclease may be an endonuclease I. An endonuclease I may be a T7 endonuclease I. Kits may include instructions for using any of the foregoing in the methods described herein.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods. Further, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art. About also includes the exact amount. Hence “about 200 nM” means “about 200 nM” and also “200 nM”.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

It is understood that the present invention is not limited to the specific details of these examples. While a preferred embodiment of the invention has been shown and described in considerable detail, it should be understood that many changes can be made in the structure without departing from the spirit or scope of the invention. Accordingly, it is not desired that the invention should be limited to the exact structure shown and described in the examples provided. As used herein, the singular forms “a,” “an,” and “the” are intended to include the plural forms as well, unless the context clearly indicates otherwise. Furthermore, to the extent that the terms “including,” “includes,” “having,” “has,” “with,” “such as,” or variants thereof, are used in either the specification and/or the claims, such terms are not limiting and are intended to be inclusive in a manner.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

### EXAMPLES

The following examples are given for the purpose of illustrating various embodiments of the invention and are not meant to limit the present invention in any fashion. It is understood that the present invention is not limited to the specific details of these examples. While a preferred embodiment of the invention has been shown and described in considerable detail, it should be understood that many changes can be made in the structure without departing from the spirit or scope of the invention. Accordingly, it is not desired that the invention should be limited to the exact structure shown and described in the examples provided.

### Example 1A: Differential Quantification Using Fragment Size Distribution

An assay may be constructed either using these probes all in different fluorescent channels, or in the same channel but at different intensity amplitudes. For example, Table 1 depicts three amplicons that may be constructed of overlapping forward/reverse regions with probe limited chemistry, such that if more than one region is amplified, the resulting detection probe intensities will add linearly. As an example, if both probe ‘A’ and ‘B’ are used in the partition, the final intensity will be a relative level of 3. In this assay, if a DNA fragment overlaps only amplicon A, a resulting intensity in the FAM fluorescent channel will be a relative level of 1 for that partition, and the fragment is guaranteed to be greater than 70 base pairs and less than 450 base pairs long, or else it would cover at least one of amplicons B or C. If a fragment of DNA is a total intensity of 2 or higher, this indicates that the fragment must be a minimum of 260 base pairs long.

TABLE 1

| | | Fwd | | Rev | | |
|---|---|---|---|---|---|---|
| | Fwd | Primer | Rev | Primer | Probe | |
| | Primer | Concen- | Primer | Concen- | Conc. + | Relative |
| Amplicon | Location | tration | Location | tration | Dye | Intensity |
| A | −35 | 1 uM | 35 | 1 uM | 250 nM | 1x |
| | | | | | FAM | |
| B | −35 | 1 uM | 225 | 1 uM | 500 nM | 2x |
| | | | | | FAM | |
| C | −225 | 1 uM | 35 | 1 uM | 500 nM | 3x |
| | | | | | FAM | |

### Example 1B: Differential Quantification Using Fragment Size Distribution—Monte Carlo Simulation

To assess the performance of the system in separating the signals of different fragment lengths of DNA, a Monte Carlo simulation was constructed. Fragments of DNA were generated by assuming a number of average coverage (e.g., 3000×) and then assigning fragments a random size and location relative to the locations of Table 1. For the purposes of this simulation, maternal DNA was selected to an average of 600 base pairs long with a standard deviation of 100 base pairs, while fetal DNA was selected to be an average of 200 base pairs long with a standard deviation of 100 base pairs. For each simulation, a ‘fetal fraction’ was chosen to determine the distribution of each size group (e.g., 10% of the DNA was assigned to the fetal size distribution). These DNA fragments were then randomly distributed among virtual reaction volumes or partitions (e.g., 20,000 partitions), and a total sum expected ‘intensity’ was assigned to each partition depending on the DNA that was present. For example, if one DNA fragment covered only amplicon A, the partition would have an intensity of 1, but if there were two fragments present that together covered A and B, the total partition intensity would be 3. Partition intensity was then randomly varied by 5% (normally distributed) noise to generate populations representative of real-world data.

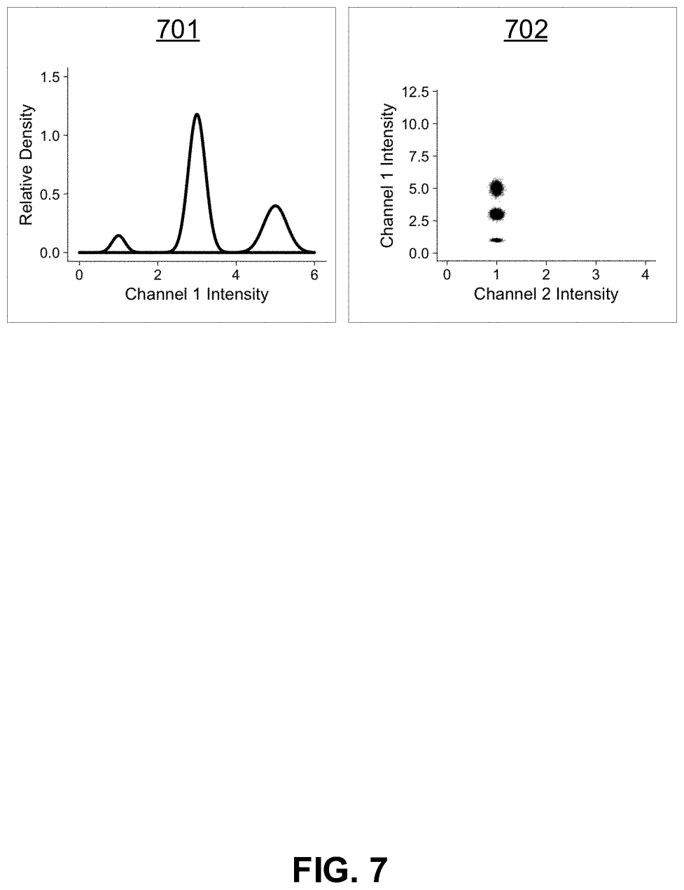

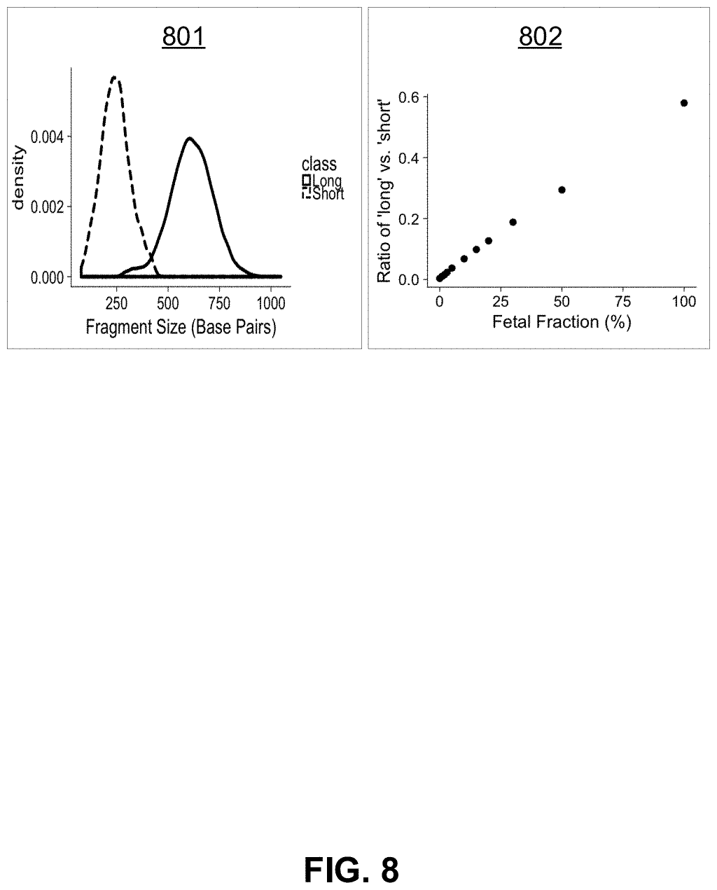

FIG. 7 illustrates, by way of example, a simulation, with a population of partitions at intensity levels of 1, 3, and 5 indicating partitions with either only A covered (intensity 1), A+B or A+C (intensity 3) or A+B+C (intensity 5) (e.g., frame 701). Classifying all partitions under intensity 2 as expected ‘short’ fragments results in clear separation of the two populations with some overlap as shown in frame 702 and in FIG. 8 at frame 801. In addition, the ratio of the number of partitions with any amplification to the number of partitions of intensity under 2 scales linearly with the fetal fraction (e.g., frame 802) which shows that a direct measurement of the distribution size and makeup is possible with this technique.

### Example 1C: Differential Quantification Using Fragment Size Distribution—NIPT Applications

Table 2 illustrates by way of example, an assay incorporating a second region from a different chromosome.

TABLE 2

| | Fwd | | Rev | | | Relative | Realtive |
|---|---|---|---|---|---|---|---|
| | Primer | Fwd Primer | Primer | Rev Primer | Probe Conc. + | Intensity | Intensity |
| Amplicon | Location | Concentration | Location | Concentration | Dye | (FAM) | (VIC) |
| A | −35 | 1 uM | 35 | 1 uM | 250 nM FAM | 1x | 2x |
| (Chr 21) | | | | | 500 nM VIC | | |
| B | −35 | 1 uM | 225 | 1 uM | 500 nM FAM | 2x | 2x |
| (Chr 21) | | | | | 500 nM VIC | | |
| C | −225 | 1 uM | 35 | 1 uM | 500 nM FAM | 2x | 2x |
| (Chr 21) | | | | | 500 nM VIC | | |
| D | −35 | 1 uM | 35 | 1 uM | 250 nM FAM | 1x | 1x |
| (Chr 18) | | | | | 250 nM VIC | | |
| E | −35 | 1 uM | 225 | 1 uM | 500 nM FAM | 2x | 1x |
| (Chr 18) | | | | | 250 nM VIC | | |
| F | −225 | 1 uM | 35 | 1 uM | 500 nM FAM | 2x | 1x |
| (Chr 18) | | | | | 250 nM VIC | | |

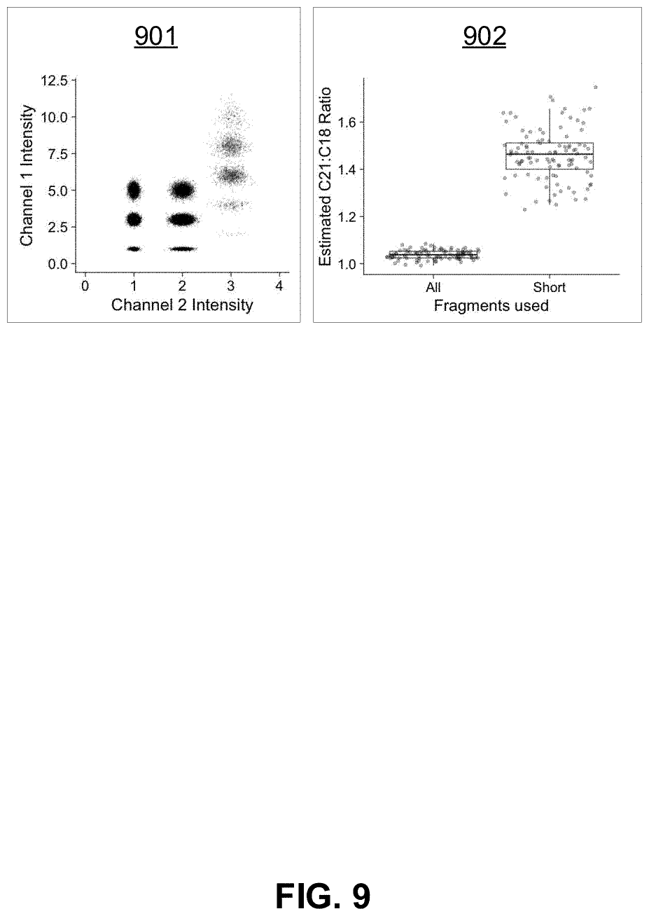

In this example, a second channel is used with different intensity levels for the different source chromosomes. In an expected NIPT application, the fetal fragment (shorter fragment) portion could have an aneuploidy such as trisomy 21, which would result in 1.5 times as much chromosome 21 as chromosome 18 in the sample of the fetal portion of the contribution. The overall ratio of the solution with a 10% fetal fraction would only be 1.05:1 Chr21:Chr18, as the maternal DNA would not be expected to have the aneuploidy. For example, frame 901 of FIG. 9 illustrates an example distribution plot of 20,000 partitions across two channels from the assay for a sample with 10% fetal fraction and trisomy 21 in the fetal portion of the DNA is shown. Frame 902 illustrates the ratio of chromosome 21 to chromosome 18 across 100 simulations for the total DNA contend results in a median close to 1.05, but with significant overlap with 1.0, which would be the expected result from a normal sample. This would indicate a test that would not be accurate in distinguishing the two conditions (normal and trisomy 21). In the ‘Short’ panel of the boxplot, the ratio between only the ‘short’ fragments has a median of 1.5, as expected from a sample that would have trisomy 21, with much greater differentiation from the baseline. The methods disclosed herein may be used in multiple applications where the fragment size of DNA present in a sample is important to measure or filter out, including the NIPT example, and also other applications, including but not limited to oncology marker measurements or STR repeat length measurements.

### Example 2A: Differential Quantification Using Single Nucleotide Polymorphism

In one example, given a mixed set of DNA which is 10% fetal fraction and an idealized SNP distribution of 5 SNPs of fetal only, 5 of maternal only, 5 of both, and 5 of neither, one will measure:

5 SNPs (fetal-only): ˜100 counts

5 SNPs (maternal-only): ˜900 counts

5 SNPs (fetal+maternal): ˜1000 counts

5 SNPs (neither): 0 counts

Here, one could then calculate the fetal fraction at 100/(100+900) counts by using the SNPs that occur only in the maternal or only in the fetal genome.

### Example 2B: Differential Quantification Using Single Nucleotide Polymorphism

In another example, the quantify of counts pertaining to maternal-only SNPs, and quantity of counts pertaining to SNPs present in both fetal and maternal sources may not be distinguishable. Thus, an adjustment can be made to the calculation to enable correct estimation of the fetal fraction without knowing, nor assigning any SNPs as maternal-only. As an example, there is an assumption of 1000 counts of genomic DNA in a solution with 20 different SNP targets. For the purposes of this example, each SNP is known to be represented in approximately 50% of the population, and furthermore, DNA consists of 10% fetal fraction with an idealized SNP distribution of 5 SNPs (fetal-only); 5 SNPs (maternal-only); 5 SNPs (fetal+maternal); and 5 SNPs (neither). Again, measure:

5 SNPs (fetal-only): ˜100 counts

5 SNPs (maternal-only): ˜900 counts

5 SNPs (fetal+maternal): ˜1000 counts

5 SNPs (neither): 0 counts

Here, where counts pertaining to maternal-only SNPs, and counts pertaining to SNPs present in both fetal+maternal sources may not be distinguishable, it may be difficult to determine the difference between the population of SNPs with 1000 counts from 900 counts. Rather, assume 5 SNPs (fetal-only): ˜100 counts and 10 SNPs (‘maternal-only’+‘fetal+maternal’) averaging 950 counts. In such a case, the maternal contribution need not be calculated, determined, or even known. Additionally, where there is a statistical probability that all 10 SNPs could be maternal-only. However, by assuming the fetal fraction is distributed relatively evenly amongst the SNPs (between the genomes), it is possible to calculate the fetal fraction as: 100/(950+100/2). This may increase the noise in the measurement as in a true sample, of the 10 SNPs identified in the upper bucket, 0, 1, 2, 3, 4, 5, 6, 7, 8, 9, or 10 of them could be maternal only due to chance.

### Example 2C: Differential Quantification Using Single Nucleotide Polymorphism—Monte Carlo Simulation

To evaluate expected ‘noise,’ a Monte Carlo simulation may be employed to model the probability of different outcomes in a simulation that cannot easily be predicted due to the intervention of random variables. This simulation assumes plasma sample from a pregnant female includes both fetal cell-free nucleic acid and maternal cell-free nucleic acid.

Here, the example simulation is set up for dPCR and includes: a single sample containing at least one nucleic acid target sequence, at least one amplification oligomer, at least one detection probe, dNTPs, a thermostable DNA polymerase, and other PCR reagents may be partitioned into approximately 20,000 evenly sized partitions. Accordingly, the Monte Carlo simulation assumes the sample would be mixed with at least a first and at least a second pair of oligonucleotide primers (wherein the first pair of oligonucleotide primers would be configured to amplify a first nucleic acid (e.g., fetal nucleic acids), and the second pair of oligonucleotide primers would be configured to amplify nucleic acid fragments of longer length (e.g., maternal nucleic acids)), and detection probes configured to detect fetal-specific SNPs; & maternal-specific SNPs. The resulting mixture would then be subjected to a dPCR.

For this example, the Monte Carlo simulation was conducted assuming 1000 copies of genomic input with various fetal fractions ranging from 2% to 30%. Then 20 SNPs were randomly assigned as fetal-only, maternal-only, fetal+maternal, or neither.

Genomic counts in each partition (absolute quantitation) were accomplished using Poisson statistical modeling. Generally, in a dPCR each partition may receive a single template of the nucleic acid target sequence. However, statistically, some partitions may receive more than one copy of a nucleic acid target template, while other partitions may not receive any target template. Poisson modeling calculates the probability of a given reaction receiving zero, one, two, three or more copies (the average number of copies per partition (λ) can be determined by λ=−ln(P(k=0)), where k is the number of positive droplets, n the total number of droplets and λ the mean copies per droplet). This ‘correction’ to enables all molecules in the starting sample to be accounted for, yielding absolute quantification. Accordingly, Poisson statistical modeling overcoming the reliance on statistical sampling (an intrinsic limitation in dPCR).

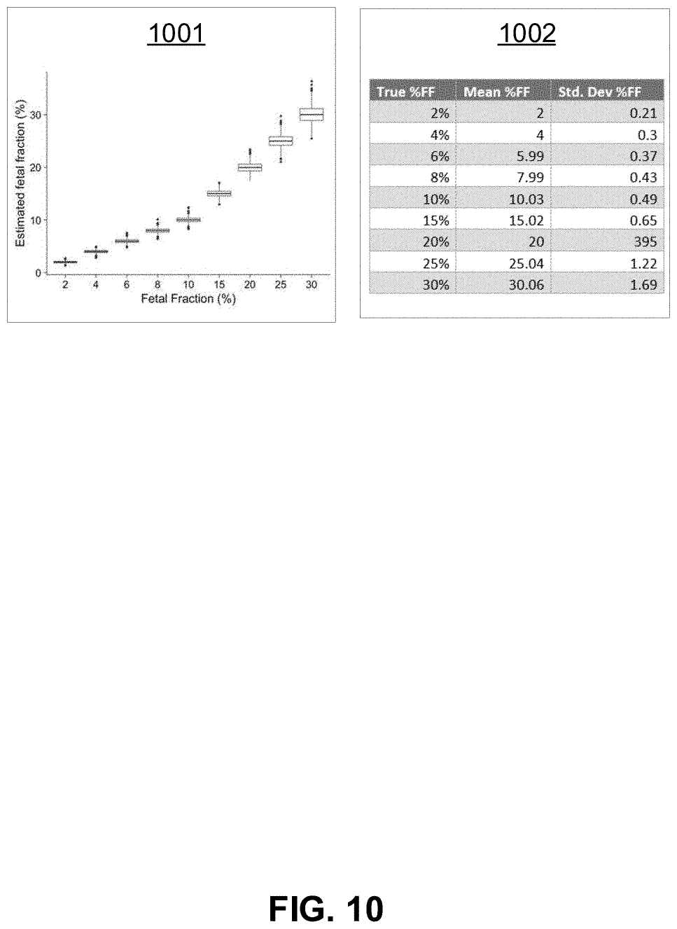

Here, 1000 simulations at each level were conducted, with the variability and statistics shown in FIG. 10 (e.g., frames 1001 and 1002). Here, 0.3% of the sample did not have a SNP of fetal-only origin would result in a ‘no call’ for that sample.

In some embodiments, in order to reduce the odds of a no-call, ‘degenerate coding’ may be employed. Degenerate coding allows for multiple SNPs to be called at each channel/intensity. In this method, multiple SNPs could be called at each channel/intensity combination. This would result in an assay where no particular SNP could be counted or called as part of the assay, but the overall counts could still be determined.

For example, 2 SNP targets (each with an allele fraction of 50%) could be used to amplify to a particular level in a particular channel. The potential number of counts determined in the previous example could be:

5 SNPs (fetal-only): ˜0, 100, or 200 counts

5 SNPs (maternal-only): ˜900, 1000, or 1100 counts

5 SNPs (fetal+maternal): ˜1800, 1900, or 2000 counts

5 SNPs (neither): 0 counts

Here, there would be a 75% chance that the sample would include a portion of its counts from the fetal fraction. Replicating this for 20 different assays, 40 SNPs provides a higher likelihood of success, whist improving the ‘no-call’ rate.

In some embodiments, employing SNP estimation may be used in situations where it is difficult to find SNP targets that are well represented across the whole population. In some embodiments, a SNP with minor allele fraction of 50% in a northern European population could be combined with a SNP with a 50% minor allele fraction from an African population to ensure proper coverage across all potential clinical sources. In these representations, averages across the pools of SNPs could be calculated and used to estimate fetal fraction, and either not all, or no individual SNPs would need to be quantitated or even the presence or absence of them identified.

What is claimed is:

1. A method for differentially quantifying nucleic acids in a biological sample using differentially methylated loci, the method comprising:
providing a solution comprising a methylation-sensitive restriction enzyme;
providing a biological sample, wherein the biological sample comprises a first plurality of nucleic acids and a second plurality of nucleic acids and wherein each of the first and second pluralities of nucleic acids further comprise a group of methylated nucleic acids and a group of unmethylated nucleic acids;
forming a digested biological sample by combining the biological sample and the solution, such that the methylation-sensitive restriction enzyme cleaves the unmethylated group of nucleic acids;
forming a reaction mixture by combining the digested biological sample with:
a set of amplification oligomers configured to amplify the first and second pluralities of nucleic acids;
a first group of detection probes configured to anneal to a region of the first plurality of nucleic acids; and
a second group of detection probes configured to anneal to a region of the second plurality of nucleic acids;
amplifying the reaction mixture to generate a first signal from the first group of detection probes and a second signal from the second group of detection probes;
determining a ratio comprising a first value derived from the first signal to a second value derived from the second signal; and
identifying the proportion of nucleic acid in the biological sample originating from the first plurality of nucleic acids.

2. The method of claim 1, further comprising partitioning the reaction mixture into reaction volumes prior to amplification.

3. The method of claim 2, wherein the first and second signals each comprise cumulative signal measurements generated from a total number of amplified reaction volumes containing an amplicon from each of the first and second pluralities of nucleic acids.

4. The method of claim 1, wherein the first signal and the second signal are each generated in a single fluorescence channel.

5. The method of claim 1, wherein the first signal and the second signal are generated in more than one fluorescence channel.

6. The method of claim 1, wherein the reaction mixture further comprises one or more of buffers, reagents, a thermostable polymerase, and deoxyribonucleotide triphosphates.

7. The method of claim 1, wherein the set of amplification oligomers comprises a forward amplification oligomer and a reverse amplification oligomer.

8. The method of claim 1, wherein the biological sample comprises genomic DNA from one or more organism.

9. The method of claim 1, wherein the first group of detection probes and the second group of detection probes each further comprise a detectable label.

10. The method of claim 9, wherein the detectable label is selected from the group consisting of a chemiluminescent label, a fluorescent label, or any combination thereof.

11. The method of claim 1, wherein the first group of detection probes and the second group of detection probes each further comprise a quencher.

12. The method of claim 1, wherein the ratio is determined without quantifying the first and second plurality of nucleic acids.

13. The method of claim 1, wherein identifying the proportion of nucleic acid in the biological sample originating from the first plurality of nucleic acids further comprises determining a fraction of methylated nucleic acids in the biological sample.

14. The method of claim 13, wherein determining a fraction of methylated nucleic acids in the biological sample comprises comparing the ratio to a reference value.

15. The method of claim 1, wherein the biological sample comprises whole blood from a pregnant female.

16. The method of claim 15, wherein the whole blood comprises maternal nucleic acids and fetal nucleic acids.

17. The method of claim 1, wherein the first plurality of nucleic acids comprises fetal nucleic acid sequences.

18. The method of claim 1, wherein the second plurality of nucleic acids comprises maternal nucleic acid sequences.

19. The method of claim 1, wherein the first group of detection probes are configured to detect a region of nucleic acid associated with fetal aneuploidy.

20. The method of claim 19, wherein the region comprises a region of chromosome 22, chromosome 21, chromosome 18, chromosome 13, chromosome 9, chromosome 8, Y chromosome, or X chromosome.

21. The method of claim 1, wherein the methylation-sensitive restriction enzyme comprises one or more of HpaII, AatII, AccII, Aor13HI, Aor51HI, BspT104I, BssHII, Cfr10I, ClaI, CpoI, Eco52I, HaeII, HhaI, MluI, NaeI, NotI, NruI, NsbI, PmaCI, Psp1406I, PvuI, SacII, SalI, SmaI and SnaBI.

22. A kit for differentially quantifying nucleic acids in a biological sample using differentially methylated loci, the kit comprising:
a methylation-sensitive restriction enzyme;
a set of amplification oligomers configured to amplify a first plurality of nucleic acids and a second plurality of nucleic acids;
a first group of detection probes configured to anneal to the first plurality of nucleic acids; and
a second group of detection probes configured to anneal to the second plurality of nucleic acids;
wherein the set of amplification oligomers comprises a forward amplification oligomer and a reverse amplification oligomer; and
wherein the first group of detection probes and the second group of detection probes each contain a detectable label.

23. The kit of claim 22, wherein the detectable label is selected from the group consisting of a chemiluminescent label, a fluorescent label, or any combination thereof.

24. The kit of claim 22, wherein the first group of detection probes and the second group of detection probes each further comprise a quencher.

25. The kit of claim 22, further comprising one or more of buffers, reagents, a thermostable polymerase, and deoxyribonucleotide triphosphates.

26. A kit for differentially quantifying a fetal fraction in a biological sample using differentially methylated loci, the kit comprising:
a methylation sensitive-restriction enzyme;
a set of amplification oligomers configured to amplify a plurality of fetal nucleic acids and a plurality of maternal nucleic acids;
a first group of detection probes configured to anneal to the plurality of fetal nucleic acids;
a second group of detection probes configured to anneal to the plurality of maternal nucleic acids;
wherein the set of amplification oligomers comprises a forward amplification oligomer and a reverse amplification oligomer; and
wherein the first group of detection probes and the second group of detection probes each comprise a detectable label.

27. The kit of claim 26, wherein the detectable label is selected from the group consisting of a chemiluminescent label, a fluorescent label, or any combination thereof.

28. The kit of claim 26, wherein the first group of detection probes and the second group of detection probes each further comprise a quencher.

29. The kit of claim 26, further comprising one or more of buffers, reagents, a thermostable polymerase, and deoxyribonucleotide triphosphates.
